(19) United States
(12) Patent Application Publication
(10) Pub. No.: US 2006/0134614 A1
(43) Pub. Date: Jun. 22, 2006

(54) Screens for altered immune response capability

(75) Inventors: Peter Beverly, Berkshire (GB); Elma Tchilian, Berkshire (GB); Tara Stanton, Oxford (GB); Walter Bodmer, Oxford (GB)

Correspondence Address: WOLF GREENFIELD & SACKS, PC;FEDERAL RESERVE PLAZA, 600 ATLANTIC AVENUE, BOSTON, MA, 02210-2206, US

(73) Assignee: The Edward Jenner Institute for Vaccine Research, Berkshire (GB)

(21) Appl. No.: 11/259,759

(22) Filed: Oct. 26, 2005

Related U.S. Application Data
(63) Continuation-in-part of application No. 10/753,754, filed on Jan. 8, 2004.
Application No. 10/753,754 is a continuation-in-part of application No. 10/020,758, filed on Oct. 30, 2001, now abandoned.

(30) Foreign Application Priority Data
Jun. 4, 2001 (GB) 0114512.7

(51) Int. Cl. C12Q 1/70; C12Q 1/68

(52) U.S. Cl. 435/5; 435/6

(57) ABSTRACT

The invention relates to associations between genetic variation in the gene encoding CD45 and human disease and human immune responses. In particular the invention provides methods of screening human subjects for the presence of an “altered immune response capability”, which may in turn affect susceptibility to viral disease and/or autoimmune disease.

### RELATED APPLICATIONS

This application is a continuation-in-part of U.S. application Ser. No. 10/753,754 filed Jan. 8, 2004, now pending, which is a continuation-in-part of U.S. application Ser. No. 10/020,758 filed Oct. 30, 2001, now abandoned, which claims priority under 35 U.S.C. §119 to GB 0114512.7, filed 14 June, 2001, the entire contents of each of which is hereby incorporated by reference.

### FIELD OF THE INVENTION

The invention relates to associations between genetic variation in the gene encoding CD45 and human disease and human immune responses. In particular the invention provides methods of screening human subjects for the presence of an “altered immune response capability”, which may in turn affect susceptibility to viral disease and/or autoimmune disease.

### BACKGROUND

The leucocyte common antigen CD45 is an abundant tyrosine phosphatase, expressed on all leucocytes (Trowbridge, I. S., and M. L. Thomas. 1994. Ann. Rev. Immunol. 12:85). The phosphatase activity of CD45 is essential for lymphocyte antigen receptor signal transduction. Both CD45 deficient mice (Kishihara, K. et al., 1993. Cell 74:143; Byth, K. et al. 1996. J. Exp. Med. 183:170) and humans (Kung, C. et al., 2000, Nature Medicine, 6: 343; Tchillan, E. Z. et al., 2001, J. Immunol., 166: 1308) are severely immunodeficient, with very few peripheral T lymphocytes and impaired T and B cell responses.

Multiple CD45 isoforms can be generated by alternative splicing of exons A, B, and C of the extracellular domain (Saga, Y. et al., 1986. Proc Natl Acad Sci USA, 83: 6940; Streuli, M. et al., 1987, J. Exp. Med., 166: 1548). In humans, naive T cells express high molecular weight CD45 isoforms, recognised by CD45RA monoclonal antibodies (mAbs), but activation of the cells results in a change to expression of low molecular weight isoforms, detected by a CD45R0 mAb (Akbar, A. N., et al., 1988, J. Immunol., 140: 2171). These two major subsets of T lymphocytes, expressing CD45RA and CD45R0 have been termed naive and memory cells, respectively.

Genetically determined abnormal CD45 splicing has been described in humans (Schwinzer, R., and K. Wonigeit, 1990, J. Exp. Med. 171:1803.). Activated or memory lymphocytes in these individuals continue to express both high and low molecular weight CD45 isoforms in contrast to the normal pattern of low molecular weight isoform expression. A C to G transversion at position 77 (C77G) in the fourth or A exon of the gene encoding CD45, has been shown to prevent the normal splicing of this exon in the affected individuals (Thude, H. et al., 1995, Eur. J. Immunol., 25: 2101; Zilch, C. F. et al., 1998, Eur. J. Immunol., 28: 22) by disrupting a strong exonic splicing silencer (Lynch, K. W. a. W., 2001, J. Biol. Chem).

The C77G polymorphism has been shown to correlate with development of multiple sclerosis in some families (Jacobsen, M. et al., 2000, Nat Genet, 26: 495), although other studies do not support such an association (Vorechovsky, I. et al., 2001, Nat Genet, 29, 22-23; Barcellos, L. F. et al., 2001, Nat Genet, 29, 23-24.

A further point mutation in exon A of CD45 (C59A) causing aberrant splicing has been identified, but appears to be relatively rare (Jacobsen, M. et al., 2002, Immunogenetics, 54, 158-163).

The present inventors have investigated the pattern of CD45 expression in HIV infection and have demonstrated a statistically significant association between the C77G mutation and HIV-1 infection.

Further observations made by the present inventors provide evidence that the C77G mutation may be a marker for general susceptibility to viral infection and/or a marker for disease severity following viral infection. Accordingly, the inventors have developed screens for determining susceptibility of human subjects to viral infection and/or identifying individuals pre-disposed to developing more severe disease following viral infection based on screening for the presence or absence of the C77G mutation at the protein, mRNA or genomic DNA level.

The present inventors have further identified a novel polymorphism A138G in exon 6 in the gene encoding CD45 with a very high prevalence in Far Eastern Oriental populations and India. The expression of various CD45 isoforms in PBMC of individuals homo- and heterozygous for the 138G variant allele was analysed and the results show that T cells in individuals carrying the 138G allele display altered cell surface CD45 isoform expression due to changes in alternative splicing. The results suggest that individuals with the 138G variant allele have an increased proportion of T cells with an activated, memory or effector phenotype, as determined by the increased proportion of CD45R0+ cells and reduced number of cells expressing the CD45 A, B and C isoforms. Analysis of exon 6 A138G and exon 4 C77G variants in different populations showed striking differences in the frequency of these mutations, suggesting effects of natural selection.

The inventors have still further identified a novel CD45 mutation, denoted A54G, in exon 4 in the gene encoding CD45. This A to G transversion results in a Thr to Ala semiconservative amino acid substitution at position 19 of the CD45RA exon 4 isoform. The A54G mutation was identified in African Ugandan populations and was found with an increased frequency amongst HIV-seropositive individuals.

The inventors findings relating to different CD45 mutations indicate that CD45 mutations can be used as genetic markers of immune function or immune capability. Furthermore, the inventors conclude that mutations in CD45 can be classified in two groups.

The first group of CD45 mutations (Group I) are associated with altered splicing of the CD45 mRNA and an altered pattern of CD45 isoform expression on the T cell population, characterized as a reduction in the proportion of T cell population carrying only the CD45R0 splice variant (i.e. a reduction in CD45R0+ T cells) in individuals carrying at least one mutant allele, as compared to individuals not carrying a mutant allele. Examples of such Group I CD45 mutations are C77G, C59A and A54G. Carriage of a mutant allele of a Group I mutation is observed to be associated with increased susceptibility to viral infection and/or a pre-disposition to developing severe disease following viral infection. Accordingly, the inventors have developed genetic screens for evaluating susceptibility to viral infection based on genotyping of Group I CD45 mutations, or on analysis of altered patterns of CD45 mRNA or protein expression associated with carriage of a Group I mutation.

The second group of CD45 mutations (Group II) are associated with altered splicing of the CD45 mRNA and an altered pattern of CD45 protein isoform expression on the T cell population, characterized as an increase in the proportion of the T cell population carrying the CD45R0 splice variant (i.e. an increase in CD45R0+ T cells) in individuals carrying at least one mutant allele, as compared to individuals not carrying a mutant allele. An example of a Group II mutation is A138G. Carriage of a mutant allele of a Group II mutation is generally observed to be associated with altered immune response capability, which may be manifest as a more vigorous response to infection by pathogenic substances or organisms, increased production of interferon-gamma by CD4 and/or CD8 T cells, an increase in the proportion of T cells of the activated, memory or effector phenotype, reduced susceptibility to viral infection and reduced susceptibility to autoimmune disease.

Accordingly, the inventors have developed screens for identifying individuals who exhibit altered immune responses based on genotyping of Group II mutations, or on screening for altered patterns of CD45 mRNA and protein expression associated with carriage of a Group II mutation. The altered immune response may affect the intensity of the immune response generated in response to exposure of an individual to pathogens. In other conditions, however, the altered immune response may be useful in treating certain diseases, for example autoimmune disorders or the like, in the event that the mutation results in an altered cytokine balance and a change in the level of response generated.

### SUMMARY OF THE INVENTION

In a first aspect the invention relates to a method of screening a human subject for susceptibility to viral infection and/or pre-disposition to developing severe disease following viral infection, which method comprises screening for the presence or absence in the genome of the subject of one or more polymorphic variants or mutations in the gene encoding CD45 or of one or more polymorphic variants in linkage disequilibrium with or in close physical proximity to a polymorphic locus in the gene encoding CD45.

In one embodiment the mutation in CD45 is characterised in that subjects carrying at least one mutant allele exhibit altered CD45 splicing resulting in an increased proportion of cells expressing CD45RA and therefore a reduced proportion of single positive CD45R0+ T cells as compared to subjects not carrying a mutant allele. In other words, carriage of a mutant allele results in a reduction in the proportion of the T cell population carrying the CD45R0 splice variant and lacking CD45RA expression (i.e. reduced proportion of single positive CD45R0+ T cells) as compared to subjects not carrying a mutant allele. Such mutations are referred to herein as “Group I” CD45 mutations. In this embodiment subjects having at least one mutant allele are scored as being more susceptible to viral infection and/or more pre-disposed to developing severe disease following viral infection, as compared to subjects who do not carry a mutant allele.

Examples of suitable Group I CD45 mutations include, but are not limited to, C77G, C59A and A54G. Screens can also be carried out using mutations or polymorphic variants in linkage disequilibrium with or close physical proximity to a Group I CD45 mutation.

In a further embodiment the mutation in CD45 is characterised in that subjects carrying at least one mutant allele exhibit altered CD45 splicing resulting in an increase in the proportion of the T cell population carrying the CD45R0 splice variant and lacking CD45RA expression (i.e. increased proportion of CD45R0+ T cells) as compared to subjects not carrying a mutant allele. Such mutations are referred to herein as “Group II” CD45 mutations. In this embodiment subjects having at least one mutant allele are scored as being less susceptible to viral infection and/or pre-disposed to developing less severe disease symptoms following viral infection, as compared to subjects who do not carry a mutant allele.

Examples of suitable Group II CD45 mutations include, but are not limited to, A138G. Screens can also be carried out using mutations or polymorphic variants in linkage disequilibrium with or close physical proximity to a Group II CD45 mutation.

In a second aspect the invention relates to a method of screening a human subject for an altered immune response capability, which method comprises screening for the presence or absence in said subject of a mutation in the gene encoding CD45, which mutation is characterised in that subjects carrying at least one mutant allele exhibit altered CD45 splicing resulting in an increase in the proportion of the T cell population carrying the CD45R0 splice variant but lacking CD45RA expression, as compared to subjects not carrying a mutant allele (i.e. a Group II CD45 mutation), wherein subjects having at least one mutant allele are scored as having altered immune response capability.

In a particular embodiment the altered immune response capability in individuals carrying a Group II CD45 mutation is manifest as a reduced susceptibility to autoimmune disease and/or reduced severity of autoimmune disease symptoms.

The invention further relates to screens based on analysis of patterns of CD45 mRNA expression or on analysis of CD45 protein isoform expression.

In further aspects the invention also provides non-human transgenic mammals which express particular combinations of CD45 isoforms. In particular the invention provides a non-human transgenic mammal (preferably a transgenic mouse). CD45^{RABC/+} mice have the genotype CD45^{+/-} at the locus encoding CD45 and also a CD45RABC transgene inserted randomly in the genome. CD45^{RO/+} mice have the genotype CD45^{+/-} at the locus encoding CD45 and also a CD45RO transgene inserted randomly in the genome. These transgenic mammals are useful models of the altered immune function in human individuals carrying mutations in CD45 and of disease severity in an autoimmune model.

### BRIEF DESCRIPTION OF THE DRAWINGS

FIG. 1 shows a digitized image of a gel and FACs analysis plots. FIG. 1A shows a gel demonstrating detection of Exon A (C77G) polymorphism. The C77G transition introduces a new restriction site for Msp I, which cleaves the mutant PCR product into two fragments of 72 and 83 bp. The presence of an undigested band of 155 bp indicates the presence of the wild type allele. FIG. 1B shows plots illustrating the results of FACS analysis to investigate the pattern of CD45 expression in human peripheral T cells pre- and post-stimulation. PBMC were stimulated with 1 μg/ml PHA and on days 0 and day 10 stained with isoform-specific CD45R0-PE and CD45RA-FITC antibody conjugates and with a CD3-APC antibody conjugate. Analysis was performed on gated CD3+ cells. Panels 1 and 2 show the normal pattern of CD45 expression pre- and post-stimulation: T cell activation is associated with a loss in CD45RA and a gain in expression of CD45R0. Panels 3 and 4 show the pattern of CD45 expression pre- and post-stimulation in a C77G heterozygote: the CD45RA population is largely absent and the T cells remain CD45RA/RO double positive after activation.

FIG. 2 shows a schematic diagram (FIG. 2A) of a family tree indicating the CD45 genotype and phenotype in each member of a family including an individual with HLH (family W). The patient with HLH (5) is indicated by an asterisk. FIG. 2A also shows a digitized image of a gel indicating the identification of the CD45 exon A (C77G) mutation in family W. The C77G transversion introduces a new restriction site for MspI, which cleaves the mutant PCR product into two fragments of 72 and 83 bp (lanes 2, 4, 5 and 6). The presence of an undigested band of 155 bp indicates the presence of the wild type allele in the father and older brother (lanes 1 and 3). FIG. 2B shows plots indicating expression of CD45 isoforms in human peripheral T cells. PBMC were stained with isoform specific CD45RA-FITC and CD45R0-PE together with CD3-APC mAbs. Analysis was performed on gated CD3+ cells. The normal pattern of CD45 isoform expression is characterised by the presence of single CD45RA+ and single CD45R0+ cells. Abnormal CD45 expression was seen in the patient (5), his mother (2) and two siblings (4 and 6). The father (1) and brother (3) have normal CD45 pattern of expression.

FIG. 3 shows a digitized image of a gel and a plot, which illustrate expression of CD45 isoforms in a patient with a common variable immunodeficiency and a history of prolonged faecal excretion of poliovirus. FIG. 3A shows the detection of exon A (C77G) polymorphism. The C77G transition introduces a new restriction site for Msp I, which cleaves the mutant PCR product into two fragments of 72 and 83 bp. The presence of an undigested band of 155 bp indicates the presence of the wild type allele. FIG. 3B shows results of flow cytometric analysis of CD45 splicing in CVID patients. Anti-CD3+ lymphocytes stained with CD45RA-FITC and CD45-RO antibodies are shown. Variant CD45 splicing in the patient with prolonged poliovirus excretion can be identified by the absence of the single CD45R0+ population.

FIG. 4 shows schematic diagrams illustrating the identification of A138G polymorphism in exon 6 of CD45. FIG. 4A shows an A→G transversion in position 138 of exon 6 that was identified. Shown are examples of a common variant homozygote allele, heterozygote and homozygotes. The changed bases are boxed. FIG. 4B provides a schematic structure of exon 6, showing the relative position of the A138G mutation, which is 7 bp from the 3′ end of exon 6. The sequence of exon 6 is shown in a one letter amino acid code and the potential glycosylation sites indicated by arrows. The mutation leads to the amino acid change 47 T→A in the coded CD45RC domain. (gcgaacacctca is SEQ ID NO:13), (gcgaacncctca is SEQ ID NO:14), (gcgaacgcctca is SEQ ID NO:15), (acagcgaacacctcaggtctga is SEQ ID NO:16), (acagcgaacgcctcaggtctga is SEQ ID NO:17), and (TANT/AS is SEQ ID NO:18).

FIG. 5 shows plots illustrating the expression of CD45 isoforms in human peripheral T cells. FIG. 5A shows results when PBMC were stained with isoform specific antibodies against CD45RA and CD45RB or CD45RA and CD45RC together with anti-CD3. Analysis was performed on gated CD3⁺ cells. A138G individuals show a decrease in the proportion of cells expressing both CD45RA and CD45RC or CD45RA and CD45RB isoforms. FIG. 5B shows results of expression of CD45R0, CD45RA and CD45RC on CD3 cells from Al 38G and control individuals. PBMC were stained with anti-CD3 together with isoform specific CD45R0 and CD45RA or CD45R0 and CD45RC antibodies. In the 138G variant, an increase in the proportion of CD45R0⁺ cells in seen. Examples are representative of similar analyses of 4A138G homozygous, heterozygous and control individuals.

FIG. 6 shows a graph and a digitized image of a gel illustrating CD45 RNA expression in PBMC from homozygous G138G and A138A individuals. Total RNA was extracted from unstimulated PBMC. After reverse transcription the resulting cDNA was amplified with primers spanning exons 2-7 of CD45 gene. PBMC from both homozygotes for G138G and common variant A138A allele individuals contained mRNA for the CD45R0 (197 bp), CD45RB (337 bp), CD45RBC (480 bp), CD45RAB (534 bp) and CD45RABC (677 bp) isoforms. FIG. 6A shows densitograms of results when bands in each lane were quantitated and shown on top of the gel corresponding to the respective isoform. The ratio between the intensity of the CD45R0 and CD45RB bands is shown at the right of the gel in FIG. 6B. Data of three representative analyses of 3 G138G homozygotes and three control samples for the common variant A138A allele are shown.

FIG. 7 shows graphs illustrating expression of CD45 isoforms and activation markers on CD4 and CD8 cells from 4 healthy G138G homozygous and 6 A138A homozygous control individuals. FIG. 7A shows the proportions of CD8 and CD4 T cells from G138G and A138A control individuals that are CD45R0+. FIG. 7B shows the proportions of CD8 and FIG. 7C of CD4 T cells that express CD11a^{hi}, CD27, CD28, CD62L, CD95 and CCR7. Means and standard deviations of data expressed as the percentage of CD8 and CD4 T cells from 4 G138G and 6 A138A control individuals are shown.

FIG. 8 shows plots illustrating expression of CD45 isoforms in peripheral T cells in Caucasian HIV seronegative (FIG. 8A) and Ugandan HIV seropositive individuals (FIG. 8B). PBMC were stained with isoform-specific CD45RA-FITC and CD45R0-PE together with CD3-APC mAbs. Analysis was performed on CD3 gated cells. The normal pattern is characterised by the presence of single positive CD45RA+ and CD45R0+ cells. Abnormal expression is seen in the C77G individual with all of the cells expressing CD45RA. In the A54G Ugandan individual there are more double positive CD45RA+R0+ cells compared to the A54A controls.

FIG. 9 shows plots of results from fluorescence activated cell sorter analysis of PBMC from 4 individuals carrying a 77G mutant allele and 4 normal control individuals (C77C homozygotes). T cells were stained for a panel of markers after gating for CD4 (FIG. 9A) and CD8 (FIG. 9B).

FIG. 10 shows graphs and a Western blot illustrating the characterisation of CD45^{RABC/+} and CD45R0^{/+} mice. FIG. 10A shows result indicating CD45 expression in CD45^{RABC/+} and CD45R0^{/+} mice. Lymph node cells were stained with pan CD45 and CD45RA isoform specific antibodies. Analysis was performed on CD3 gated cells. The shaded histogram is CD45^{+/+}, dotted line CD45^{RABC/+}, solid CD45R0^{/+} and the grey dotted isotype control. Examples are representative of three mice of each type. FIG. 10B shows results indicating activation of T cells from CD45^{+/+}, CD45^{RABC/+} and CD45R0^{/+} mice. Mesenteric and peripheral lymph node cells were activated in the presence of varying amounts of plate bound CD3 and 1 μg/ml of CD28 antibodies. Cells were pulsed with [³H] thymidine at 24, 48, 72 and 96 hours and harvested after 12 h. Means and standard deviations of triplicate cultures from three mice are shown. Background counts were less than 500 cpm and have been subtracted. Data are representative of 5 experiments. FIG. 10C shows results of annexin staining of cells cultured in the presence of 2 μg/ml CD3 and 1 μg/ml CD28 at the indicated time points. Means and standard deviations of triplicate cultures from three mice are shown. FIG. 10D is a Western blot of Lck and pY505Lck in lymph node T cells stimulated with 2 μg/ml CD3 and 1 μg/ml CD28 for the times indicated. Data are representative of 4 experiments.

FIG. 11 shows graphs and Western blots illustrating cytokine production and response of CD45^{+/+}, CD45^{RABC/+} and CD45R0^{/+} mice. FIGS. 11A-B show results when lymph node cells were stimulated for 72 hours with CD3/CD28 (FIG. 11A) or PMA-ionomycin (FIG. 11B). The histograms show means and standard deviation of the amounts in supernatants of triplicate cultures from 3 mice. Results are representative of 6 experiments. FIG. 11C shows results indicating activation of lymph node T cells with PMA-ionomycin. Cells were pulsed with [³H] Thymidine at 72 hrs and harvested 12 hrs later. Means and standard deviations of triplicate cultures from three mice are shown. FIG. 11D is a Western blot of STAT1 and pY701STAT1 in lymph node T cells stimulated with PMA-ionomycin for the times indicated. Data are representative of 3 experiments.

FIG. 12 is a graph of results indicating experimental autoimmune encephalomyelitis (EAE) in CD45^{+/+}, CD45^{RABC/+} and CD45R0^{/+} mice. EAE was induced in groups of 5-9 mice of each strain and disease monitored as described in Methods. The graph represent the mean score for each day from three experiments.

### DEFINITIONS

In the context of this application the terms “gene encoding CD45” and “CD45 gene” are used interchangeably and refer to a gene, also referred to as the PTPRC gene, located at gene map locus 1q31-32 (OMIM accession 151460). The complete sequence of the gene is available via publicly accessible genome sequence databases. A list of GenBank accession numbers for individual exons of the gene is provided.

The terms “C77G polymorphism”, “A138G polymorphism”, “C59A polymorphism” and “A54G polymorphism” may be used herein to refer to the respective polymorphic loci.

When referring to individual alleles of the C77G polymorphism in exon 4 of the CD45 gene, the terms “mutant allele”, “variant allele”, “C77G variant” and “C77G mutation” should be taken to refer to the 77G allele, i.e. the allele having G at position 77. The terms “normal allele” and “wild type allele” should be taken to refer to the 77C allele, i.e. the allele having C at position 77.

When referring to the carrier status of individual human subjects the term “G77G” refers to an individual homozygous for the 77G allele, the term “C77C” refers to an individual homozygous for the 77C allele and the term “C77G” refers to a heterozygous individual.

The terms “carrier(s) of the 77G allele” and “individual(s) having the 77G variant” refer to both homozygotes for 77G and heterozygotes.

The terms “individual having the C77G variant” or “individual having the C77G mutation” may, depending on the context in which it is used, also refer to any individual having a 77G allele, i.e. encompassing both homozygotes for 77G and heterozygotes.

When referring to individual alleles of the A138G polymorphism in exon 6 of the CD45 gene, the terms “mutant allele”, “variant allele”, “A138G variant” and “A138G mutation” should be taken to refer to the 138G allele, i.e. the allele having G at position 138. The terms “normal allele” and “wild type allele” should be taken to refer to the 138A allele, i.e. the allele having A at position 138.

When referring to the carrier status of individual human subjects the term “G138G” refers to an individual homozygous for the 138G allele, the term “A138A” refers to an individual homozygous for the 138A allele and the term “A138G” refers to a heterozygous individual.

The terms “carrier(s) of the 138G allele” and “individual(s) having the 138G variant” refer to both homozygotes for 138G and heterozygotes.

The terms “individual having the A138G variant” or “individual having the A138G mutation” may, depending on the context in which it is used, also refer to any individual having a 138G allele, i.e. encompassing both homozygotes for 138G and heterozygotes.

When referring to individual alleles of the C59A polymorphism in exon 4 of the gene encoding CD45, the terms “mutant allele”, “variant allele”, “C59A variant” and “C59A mutation” should be taken to refer to the 59A allele, i.e. the allele having A at position 59. The terms “normal allele” and “wild type allele” should be taken to refer to the 59C allele, i.e. the allele having C at position 59.

When referring to individual alleles of the A54G polymorphism in exon 4 of the gene encoding CD45, the terms “mutant allele”, “variant allele”, “A54G variant” and “A54G mutation” should be taken to refer to the 54G allele, i.e. the allele having G at position 54. The terms “normal allele” and “wild type allele” should be taken to refer to the 54A allele, i.e. the allele having A at position 54.

The protein encoded by the human CD45 gene exists in multiple isoforms, depending on alternative splicing of exons 4, 5 and 6. “CD45RA” refers to isoforms containing the CD45RA domain encoded by exon 4, “CD54RB” refers to isoforms containing the CD45RB domain encoded by exon 5 and “CD45RC” refers to isoforms containing the CD45RC domain encoded by exon 6, whereas “CD45R0” refers to a low molecular weight isoform which lacks exons 4-6. Where a cell or tissue is referred to herein as “lacking expression” of a particular CD45 isoform this may be taken to mean that substantially no expression of the protein isoform is detectable using standard techniques for analysis of protein expression, for example FACs analysis, Western blotting etc. Where a cell or tissue is referred to herein as “lacking expression” of mRNA encoding a CD45 isoform, this may be taken to mean that substantially no expression of the mRNA is detectable using standard techniques for analysis of mRNA expression, for example RT-PCT, RNase protection, Northern blotting etc.

The term “non-human mammal” is taken to include inter alia rodents, non-human primates, sheep, dogs, cows, pigs and cats. Preferred non-human mammals are selected from the rodent family including rat and mouse, most preferably mouse.

The term “transgene” means a polynucleotide sequence comprising a coding region which encodes a polypeptide (e.g. encoding a single CD45 isoform) that has been introduced into at least one cell of a transgenic organism by way of human intervention. A transgene may be partly or entirely heterologous, i.e. foreign to the transgenic organism into which it is introduced. The transgene may include transcriptional regulatory sequences and any other sequences, e.g. introns, which may be useful for optimal expression of the coding sequence.

The term “transgenic mammal” includes any non-human mammal which expresses a transgene in one or more cells or tissues, the transgene having been introduced by way of human intervention, such as by transgenic techniques well known in the art.

### DETAILED DESCRIPTION OF THE INVENTION

The invention provides genetic screens based on genotyping of genetic variants or mutations in the CD45 gene for determining susceptibility of human subjects to viral infection and/or autoimmune disease, and/or for identification of subjects having “altered immune response capability”.

As will be illustrated in the accompanying Examples, a mutation (C to G transversion) at position 77 in the fourth or “A” exon of the CD45 gene has been shown to be associated with HIV-1 infection. In addition, the C77G mutation has been found in a patient with common variable immunodeficiency with persistent viral infection and prolonged excretion of polio virus (this patient was previously described by Misbah et al., Postgrad Med J, 1991, Vol: 67, 301-303; see Example 3) and in a patient infected with EBV (data not shown). Furthermore, the inventors have shown the C77G mutation to be present in patients diagnosed with haemophagocytic lymphohistiocytosis (HLH) (see Example 2). Sporadic cases of HLH are often provoked by viral infection in childhood (Dreyer, et al., Am J Pediatr Hematol Oncol, Vol: 13, 476). The inventors have still further shown that the 77G variant allele is present with an increased frequency in patients with a chronic hepatitis C virus (HCV) infection who were not able to clear the virus, compared to HCV infected individuals who resolve the infection and become HCV RNA virus negative.

As further illustrated in the examples provided, a mutation (A to G transversion) at position 138 in exon 6 of the CD45 gene, has been shown to be associated with an alteration in cell surface CD45 isoform expression, the transversion resulting in a Threonine to Alanine semi-conservative amino acid change at position 47 of the CD45RC exon 6. The mutation causes dramatic changes in the proportions of T cells expressing different CD45 isoforms, with individuals having the 138G variant having an increased proportion of T cells with an activated, memory or effector phenotype, as determined by an increased proportion of CD45R0+ cells, and reduced numbers of cells expressing the CD45 A, B and C isoforms. G138G homozygotes also exhibit altered expression of other leukocyte antigens, namely decreased expression of CD27, CD28, CD62L and CCR7 and increased expression of CD11a and CD95. These changes again indicate that the most prominent effect in 138G individuals is an increase in the proportion of activated/memory T cells having the CD45R0+ phenotype.

As aforesaid, the inventors have concluded that mutations in CD45 can be classified in two groups, as follows:

Group I CD45 mutations, exemplified by C77G and C59A, are associated with altered splicing of the CD45 mRNA and an altered pattern of CD45 isoform expression on the T cell population, characterized as a reduction in the proportion of T cell population carrying only the CD45R0 splice variant (i.e. a reduction in CD45R0+ T cells) in individuals carrying at least one mutant allele, as compared to individuals not carrying a mutant allele. Carriage of a mutant allele of a Group I mutation is generally observed to be associated with increased susceptibility to viral infection and/or a pre-disposition to developing severe disease following viral infection.

Group II CD45 mutations, exemplified by A138G, are associated with altered splicing of the CD45 mRNA and an increase in the proportion of the T cell population having the CD45R0+ phenotype in individuals carrying at least one mutant allele, as compared to individuals not carrying a mutant allele. Carriage of a mutant allele of a Group II mutation is generally observed to be associated with altered immune response capability, which may be manifest as a more vigorous response to infection by pathogenic substances or organisms, increased production of interferon-gamma by CD4 and/or CD8 T cells, an increase in the proportion of T cells of the activated, memory or effector phenotype, reduced susceptibility to viral infection and reduced susceptibility to autoimmune disease.

Genetic Screens Based on Group I Mutations

Genetic screens based on genotyping of one or more Group I CD45 mutations may be used to screen human subjects for susceptibility to viral infection and/or pre-disposition to developing severe disease following viral infection. Individuals having at least one mutant Group I allele are scored as being more susceptible to viral infection and/or pre-disposed to developing severe disease following viral infection.

A “mutant Group I CD45 allele” is defined as a mutant allele of CD45, carriage of which causes (or is associated with) altered splicing of the CD45 mRNA, preventing splicing out of exon 4 of CD45, and an altered pattern of CD45 protein isoform expression manifest as a reduction in the proportion of the T cell population having the CD45R0+ phenotype. Activated/memory T lymphocytes in individuals carrying a Group I mutation continue to express both CD45RA and CD45R0 isoforms, in contrast to the “normal” pattern of low molecular weight CD45R0 expression (see accompanying examples).

In particular non-limiting embodiments of the invention screens based on Group I mutations (or polymorphic variants in linkage disequilibrium with or close physical proximity to) a Group I mutation may be used to screen for susceptibility to infection with viruses selected from the group consisting of human immunodeficiency viruses, and in particular HIV-1, Epstein-Barr virus, poliovirus, hepatitis B virus (HBV) and hepatitis C virus (HCV).

The methods of the invention preferably comprise genotyping of one or more Group I CD45 mutations which have previously been demonstrated to show statistically significant genetic association with susceptibility to viral disease and/or severity of viral disease, for example in a population-based genetic association study or case-controlled study. However, the utility of the invention is not strictly limited to mutations for which a statistically significant disease association has been demonstrated by experiment, since it is possible to predict disease association on the basis of classification as a Group I or Group II mutation.

Suitable Group I mutations include, but are not limited to, C77G, C59A and A54G. Individuals carrying at least one mutant allele of either mutation (i.e. 77G or 59A) are scored as being susceptible to viral infection and/or pre-disposed to developing severe disease following viral infection, as compared to individuals not carrying a mutant allele.

The invention also contemplates screens based on polymorphic variants or mutations (whether or not within the CD45 gene) which have not themselves been shown to be genetically associated with susceptibility to viral infection and/or severity of disease in a population-based study but which are either in linkage disequilibrium with or in close physical proximity to a Group I (and/or a Group II) mutation in CD45.

As would be readily apparent to persons skilled in the art of human genetics, “linkage disequilibrium” occurs between a marker polymorphism (e.g. a DNA polymorphism which is “silent” and a functional polymorphism (i.e. genetic variation which affects phenotype or which contributes to a genetically determined trait) if the marker is situated in close proximity to the functional polymorphism. Due to the close physical proximity, many generations may be required for alleles of the marker polymorphism and the functional polymorphism to be separated by recombination. As a result they will be present together on the same haplotype at higher frequency than expected, even in very distantly related people.

As used herein the term “close physical proximity” means that the two markers/alleles in question are close enough for linkage disequilibrium to be likely to arise. In one embodiment the polymorphic variant in “close physical proximity” to a Group I mutation in CD45 will be located within a genetic distance of 1 cM or less from the Group I mutation. In another embodiment the polymorphic variant in “close physical proximity” to a Group I mutation in CD45 will be located within a physical distance of less than 1 megabase, or less than 500 kB, or less than 200 kB, or less than 100 kB, or less than 50 kB, or less than 20 kB, or less than 10 kB, or less than 5 kB of the Group I mutation. The polymorphic variant in “close physical proximity” of the Group I mutation may be within the CD45 gene but this is not essential; it may be located within a different gene or within an intergenic region of the genome.

In such screens individuals carrying at least one allele in linkage with or close physical proximity to a Group I mutant allele will be scored as being susceptible to viral infection and/or pre-disposed to developing severe disease following viral infection.

Genetic Screens Based on Group II Mutations

Genetic screens based on genotyping of one or more Group II CD45 mutations may be used to screen human subjects for “altered immune response capability”. Individuals having at least one Group II mutant allele are scored as having altered immune response capability or altered immunological function as compared to individuals not having a mutant allele.

A “mutant Group II CD45 allele” is defined as a mutant allele of CD45, carriage of which causes (or is associated with) altered splicing of CD45 mRNA which is characterised as a quantitative increase in the level of expression of the CD45R0 transcript and an altered pattern of CD45 protein isoform expression manifest as an increase in the proportion of the T cell population having the CD45R0+ phenotype.

Suitable Group II mutations include, but are not limited to, A138G. Individuals carrying at least one mutant allele (i.e. 138G) are scored as having altered immune response capability/altered immunological function, as compared to individuals not carrying a mutant allele.

The invention also contemplates screens based on polymorphic variants or mutations (whether or not within the CD45 gene) which have not themselves been shown to be associated with altered immune response capability or altered immunological function in a population-based study but which are either in linkage disequilibrium with or in close physical proximity to a Group II mutation in CD45, e.g. A138G. The use of alleles in linkage disequilibrium with or in close physical proximity to a Group II mutation applies to all specific embodiments of the genetic screens based on Group II mutations described herein. In this context the terms “linkage disequilibrium” and “close physical proximity” are to be given the same meaning as that given above in relation to Group I CD45 mutations.

In one embodiment the “altered immune response capability” associated with carriage of a Group II mutation such as the 138G allele may be defined as an increase in the proportion of T cells having the activated, memory or effector phenotype, as determined by an increase in the proportion of CD45R0+ T cells, as compared to control individuals homozygous for the “wild type” allele (e.g. 138A). Quantitative analysis of the relative proportions of CD45R0+ positive T cells versus T cells expressing CD45RA, RB or RC isoforms can be carried out using any suitable technique known in the art, such as for example FACS analysis, as illustrated in the accompanying Examples.

T cells having the activated, memory or effector phenotype may be identified on the basis of expression patterns for certain marker proteins. In particular, “activated” T cells may be characterized by decreased expression of CD62L and increased expression of CD11a and CD95, as compared to naive T cells. In individuals carrying Group II CD45 mutations, such as 138G, activated T cells generally express the CD45R0 isoform, whereas in individuals carrying Group I mutations, such as 77G, as significant proportion of activated T cells express both CD45RA and CD45R0 isoforms. However, in both Group I and Group II mutant carriers “activated” T cells can be identified/characterized on the basis of expression levels of CD62L, CD11a and CD95.

The inventors have further shown that carriage of a Group II CD45 mutation, such as the 138G allele, is associated with increased production of the Th1 cytokine interferon (IFN) gamma by CD4 and CD8 T cells. This provides strong support for the link between carriage of Group II mutations such as the 138G allele and altered immune response capability/altered immune function.

Therefore, in a further embodiment of the invention the “altered immune response capability” associated with carriage of a Group II mutant allele, such as the 138G allele, may be defined as increased production of IFN-gamma by CD4 and/or CD8 T cells, as compared to individuals homozygous for the equivalent wild type allele (e.g. 138A).

The finding that carriage of a Group II mutant allele, such as the 138G allele, is associated with an increase in the proportion of T cells having the activated, memory or effector phenotype and with increased production of the Th1 cytokine IFN-gamma by CD4 and CD8 T cells means that genetic screening of a human subject for carriage of a Group II mutant allele, such as 138G, can provide a useful indication of the immune capability of that subject. Thus, genetic screening for a Group II mutant allele, such as 138G, may potentially be used to evaluate susceptibility of a human subject to (i) any disease wherein disease pathogenesis is affected (either positively or negatively) by the production of an increased proportion of activated, memory or effector T cells, and (ii) any disease wherein disease pathogenesis is affected (either positively or negatively) by increased production of IFN-gamma. Moreover, genetic screening for a Group II mutant allele, such as 138G, may also potentially be used to evaluate likely severity of disease symptoms for (i) any disease in which the severity of disease symptoms is affected (either positively or negatively) by the production of an increased proportion of activated, memory or effector T cells, and (ii) any disease in which the severity of disease symptoms is affected (either positively or negatively) by increased production of IFN-gamma. The practical applications of screens based on genotyping of Group II mutations such as the A138G polymorphism are therefore potentially very wide within the spectrum of infectious, immune and autoimmune diseases. Susceptibility to disease and/or likely severity of disease will be evaluated based on the presence or absence of the Group II mutant allele (e.g. 138G), depending on whether production of an increased proportion of activated, memory or effector T cells and/or increased production of IFN-gamma is a positive or negative factor from the perspective of the subject under test, i.e. whether these factors promote susceptibility or resistance to the disease in question, or promote severe or mild symptoms of the disease in question.

The increased effector T cell population and/or increased production of the Th1 cytokine IFN-gamma by CD4 and CD8 T cells in individuals having Group II mutations such as the 138G variant may lead to a more vigorous immune response to pathogens. The inventors have shown in the accompanying examples a significant dominant protective effect for the 138G allele in infection with hepatitis B virus (HBV) and also a decreased frequency of the 138G allele in individuals with chronic hepatitis C virus (HCV) infection.

Therefore, in a still further embodiment of the invention the “altered immune response capability” associated with carriage of a Group II mutation such as the 138G allele may be defined as a more vigorous response to pathogenic substances or pathogenic organisms, as compared to individuals homozygous for the wild type allele (e.g. 138A).

In a preferred embodiment the pathogenic organism may be a virus. Genotyping of individuals for a Group II mutation such as the A138G polymorphism may therefore be used to provide an indication of susceptibility to viral infection and/or an indication of the likely severity of disease symptoms following viral infection. In a specific embodiment the viral infection may be infection with hepatitis B virus or hepatitis C virus. In these embodiments, the presence of at least one Group II mutant allele (e.g. 138G) will be taken as an indication of reduced susceptibility to viral infection and/or reduced severity of disease following infection, as compared to individuals homozygous for the wild type allele (e.g. 138A). Such genetic screens might be used, for example, to screen uninfected individuals or those in a very early stage of viral infection in order to evaluate whether the individual is susceptible to viral infection or is pre-disposed, by virtue of their genetic make-up, to develop more or less severe disease symptoms following viral infection, particularly infection with HBV or HCV. This knowledge might be useful, for example, in the selection of appropriate treatment (including prophylaxis) for particular individuals. The screens may be of particular use in the screening of neonates and infants in order to determine susceptibility to HBV or HCV infection and/or likely severity of disease following infection with HBV or HCV, as well as predicting response to vaccination in both neonates and adults.

Genotyping of Group II mutations, e.g. A138G, may be used to provide an indication of susceptibility to viral infection and/or an indication of the likely severity of disease symptoms following viral infection in (i) any viral infection wherein susceptibility to infection and/or the severity of disease symptoms is affected (either positively or negatively) by the production of an increased proportion of activated, memory or effector T cells, and (ii) any viral infection wherein susceptibility to infection and/or the severity of disease symptoms is affected (either positively or negatively) by increased production of IFN-gamma.

IFN-gamma has been shown to be of crucial importance in protective immunity against many infectious diseases, including hepatitis B itself (see Viral pathogenesis Chapter 31 viral hepatitis—Francis V. Chisari and Carlo Ferrari pg 745-778 Editor in chief—Neal Nathanson 1997 Lippincott-Raven publishers, 227 East Washington Square, Philadelphia. A 19106) and tuberculosis. Genetic screens for carriage of the 138G allele can provide a useful indication of disease susceptibility and/or likely disease severity for any infectious disease in which IFN-gamma provides/promotes protective immunity. Other references in the art to the role of IFN-gamma are as follows: Immunobiology: the immune system in health and disease 5th edition Published in 2001 by garland publishing, a member of the Taylor and Francis Group, 29 West 35th Street, New York N.Y.; Charles A. Janeway, Paul Travers, Mark Walport, Mark J. Shlomchik, Szabo S J, Sullivan B M, Peng S L, Glimcher L H. Molecular mechanisms regulating Th1 immune responses. Annu Rev Immunol. 2003;21:713-58. Epub Dec. 19, 2001.; Vandenbroeck K, Goris A Cytokine gene polymorphisms in multifactorial diseases: gateways to novel targets for immunotherapy?. Trends Pharmacol Sci. June 2003;24(6):284-9.; Adorini L. Cytokine-based immunointervention in the treatment of autoimmune diseases. Clin Exp Immunol. May 2003;132(2):185-92.; Factor P. Gene therapy for asthma. Mol Ther. February 2003;7(2):148-52.; Chesler D A, Reiss C S. The role of IFN-gamma in immune responses to viral infections of the central nervous system. Cytokine Growth Factor Rev. December 2003;13(6):441-54.

In some conditions the increased effector T cell population and/or the increased production of the Th1 cytokine IFN-gamma by CD4 and CD8 T cells in individuals having a Group II mutation such as 138G may be a negative regulator, contributing to the cessation of the immune response, or resulting in a less vigorous or altered immune response, which may be less pathogenic in the individuals who develop the disease or which may reduce the risk of autoimmune disease in carriers of the mutation. In particular, the inventors have shown in the accompanying examples a significant dominant protective effect for the 138G allele in the autoimmune disorder Grave's disease, a recessive protective effect for the 138G allele in the autoimmune disease Hashimoto's thyroiditis and a decreased frequency of the 138G allele in type I diabetes.

Therefore, in a still further embodiment of the invention the “altered immune response capability” associated with carriage of a Group II mutation such as the 138G allele may be defined as a less vigorous or pathogenic immune response to autoantigen, as compared to individuals homozygous for the wild type allele (e.g. 138A).

Accordingly, genotyping of an individual for a Group II mutation such as the A138G polymorphism may be used to evaluate susceptibility to autoimmune disease and/or as an indicator of the likely severity of autoimmune disease in the individual. In a specific embodiment the autoimmune disease may be Grave's disease, Hashimoto's thyroiditis or type I diabetes. In these embodiments, the presence of at least one Group II mutant allele (e.g. 138G) will be taken as an indication of reduced susceptibility to autoimmune disease and/or reduced severity of autoimmune disease symptoms, as compared to individuals homozygous for the equivalent wild type allele (e.g. 138A). Such genetic screens might be used, for example, to screen asymptomatic individuals thought to be “at risk” of developing autoimmune disease, or individuals manifesting very early symptoms of the disease in order to evaluate whether the individual is pre-disposed, by virtue of their genetic make-up, to develop more or less severe disease symptoms. This knowledge might be useful, for example, in the selection of appropriate treatment (including prophylaxis) for that individual.

An “autoimmune disease” may be defined as a disease in which there is sustained cellular and/or humoral autoreactive immunity and evidence for a pathogenic role of the autoreactive cells or antibodies.

Genotyping for a Group II mutation, e.g. A138G, may be used to evaluate susceptibility to autoimmune disease and/or as an indicator of the likely severity of autoimmune disease in (i) any autoimmune disease wherein susceptibility to the disease and/or the severity of disease symptoms is affected (either positively or negatively) by the production of an increased proportion of activated, memory or effector T cells, and (ii) any autoimmune disease wherein susceptibility to the disease and/or the severity of disease symptoms is affected (either positively or negatively) by increased production of IFN-gamma.

The increased effector T cell population and/or increased production of the Th1 cytokine IFN-gamma by CD4 and CD8 T cells in individuals having the 138G variant may still further confer resistance to atopic diseases and allergy. Accordingly, screens for carriage of a Group II mutation such as the 138G allele may further provide useful prognostic information relating to atopic and allergic diseases.

“Allergy” and “atopy” are conditions in which the immune system responds excessively and/or inappropriately to antigens so that tissue damage or other symptoms may result. Immmune responses in these conditions are usually Th2 biased resulting in the production of IgE antibody and in the presence of antigen, the activation of basophils and mast cells and release of histamine and other mediators. Examples of allergy reactions are the acute anaphylactic response of some individuals to bee venom or grass pollens, while dermatitis or asthma represent atopic disorders.

Genotyping of a Group II mutation such as the A138G polymorphism in individuals either “at risk” of developing allergy/atopy or those already manifesting disease symptoms may be useful in selecting appropriate treatment regimes. For example, individuals who develop early disease, e.g. asthma, and are at higher risk because they lack a Group II mutation such as the 138G allele might be scored as candidates for more vigorous early therapy to prevent chronic and more severe disease developing. There is evidence that early BCG immunization, a Th1 stimulus, is associated with protection against subsequent development of allergy in Japan (Shirakawa T., Enomoto T., Shimazu S. & Hopkins J. M. (1997) The inverse association between tuberculin responses and atopic disorder. Science, 275, 77) so one might predict that Group II mutations such as 138G would be similarly protective.

Genetic screens for carriage of Group II mutations such as 138G may be used diagnostically and/or prognostically, depending on the nature of the disease/condition which it is desired to evaluate, and/or on the status of the patient/subject under test. However, the actual screening methodology will generally be the same regardless of whether the screen is used diagnostically or prognostically. An extremely useful application of the genetic screens is likely to be in predicting the likely outcome of a particular course of treatment/therapy in a given individual, depending on carrier status for the Group II mutation (e.g. 138G). The genetic screens may still further be useful in predicting who will develop disease complications, such as, for example, carcinoma following infection with HBV, or nasopharyngeal carcinoma in EBV infection and adult T cell leukemia or HTLV-1 associated myelopathy/tropical spastic paraparesis (HAM/TSP) in HTLV-1 disease.

Genetic screens based on genotyping of Group II mutations such as A138G may also be used in order to predict the likely response of an individual to a vaccine. The “altered immune response capability” associated with carriage of a Group II mutation in a human subjects may affect the ability of an individual to mount an immune response to a challenging antigen or vaccine. Thus, the genetic screens may be used to predict whether vaccination is likely to be successful in a given subject.

In the case of vaccines which induce a protective antibody response, the “altered immune response capability” associated with carriage of the 138G allele will generally pre-dispose to a less strong response, because Th2 cytokines are needed to stimulate production of high antibody titres. Typing for A138G may therefore be used to predict poor vaccine responders who might need an extra boost in order to achieve protection.

In the case of vaccines which induce cellular immunity (Th1 mediated), carriage of 138G will generally pre-dispose to development of a stronger response.

In a particular embodiment the genetic screens may be used to predict the likely response to anti-tumour vaccines. In the case of anti-tumour vaccines that induce a Th1 cellular response, carriers of the 138G allele may be scored as likely to exhibit a more positive response that individuals who do not carry 138G.

General Genotyping Methodology

In the context of the invention, the process of screening for the presence or absence of a mutation or allelic variant in the genome of an individual may advantageously comprise screening for the presence or absence in the genome of the subject of both the common or wild type allele and the variant or mutant allele or may comprise screening for the presence or absence of either individual allele, it generally being possible to draw conclusions about the genotype of an individual at a polymorphic locus having two alternative allelic forms just by screening for one or other of the specific alleles.

The step of screening for the presence or absence of a mutation or allelic variant in the genome of a subject, also referred to herein as “genotyping”, can be carried out using any suitable methodology known in the art and it is to be understood that the invention is in no way limited by the precise technique used to perform such genotyping.

Known techniques for the scoring of single nucleotide polymorphisms include, but are not limited to, mass spectrometry, particularly matrix-assisted laser desorption/ionization time-of-flight mass spectrometry (MALDI-TOF-MS), single nucleotide primer extension and DNA chips or microarrays (see review by Schafer, A. J. and Hawkins, J. R. in Nature Biotechnology, Vol 16, pp 33-39 (1998)). The use of DNA chips or microarrays may enable simultaneous genotyping at many different polymorphic loci in a single individual or the simultaneous genotyping of a single polymorphic locus in multiple individuals. SNPs may also be scored by DNA sequencing using any known sequencing methodology.

In addition to the above, SNPs are commonly scored using PCR-based techniques, such as PCR-SSP using allele-specific primers (described by Fanning, G. C., et al., Tissue Antigens, 1995; 50: 23-31). This method generally involves performing DNA amplification reactions using genomic DNA as the template and two different primer pairs, the first primer pair comprising an allele-specific primer which under appropriate conditions is capable of hybridising selectively to the wild type allele and a non allele-specific primer which binds to a complementary sequence elsewhere within the gene in question, the second primer pair comprising an allele-specific primer which under appropriate conditions is capable of hybridising selectively to the variant allele and the same non allele-specific primer. Still further PCR-based techniques for scoring SNPs include PCR ELISA, AMDI and DHPLC.

If the SNP results in the abolition or creation of a restriction site, as is the case with the C77G mutation in the CD45 gene, genotyping can be carried out by performing PCR using non-allele specific primers spanning the polymorphic site and digesting the resultant PCR product using the appropriate restriction enzyme (also known as PCR-RFLP). Restriction fragment length polymorphisms, including those resulting from the presence of a single nucleotide polymorphism, may be scored by digesting genomic DNA with an appropriate enzyme then performing a Southern blot using a labelled probe corresponding to the polymorphic region (see Molecular Cloning: A Laboratory Manual, Sambrook, Fritsch and Maniatis, Cold Spring Harbor Laboratory, Cold Spring Harbor, N.Y.).

The known techniques for scoring polymorphisms are of general applicability and it will be readily apparent to persons skilled in the art that known techniques may be adapted for the scoring of single nucleotide polymorphisms in the CD45 gene.

In the case of the C77G mutation, a suitable technique for genotyping of this single mutation is PCR followed by digestion of the PCR product with the enzyme MspI, as described in the accompanying Example. However, the invention is not intended to be limited to the use of this technique.

In the case of the A138G mutation, one suitable technique for genotyping of this single mutation is ARMS PCR, as illustrated in the accompanying Examples. However, the invention is not intended to be limited to the use of this technique.

In further non-limiting embodiments, both the Al 38G and C77G mutations can be typed using a Sequenom Mass-Array® MALDI-TOF primer extension assay. The general features of this assay are described in Jurinke et al. The use of MassARRAY technology for high throughput genotyping. Adv Biochem Eng Biotechnol. 2002;77:57-74 (see also www.sequenom.de/).

Genotyping is preferably carried out in vitro, and is most preferably performed on isolated genomic DNA prepared from a suitable tissue sample obtained from the subject under test. Most commonly, genomic DNA is prepared from a sample of whole blood, according to standard procedures, which are well known in the art.

Most advantageously, it is envisaged that individuals will be simultaneously genotyped for multiple CD45 mutations in order to provide a “profile” of overall immune capability/disease susceptibility. Simultaneous genotyping at multiple loci may be achieved, for example, with the use of “gene chips” or microarrays. It is also contemplated that genotyping of CD45 mutations may be carried out simultaneously with genotyping of polymorphic variants/mutations in genes other than CD45 that are also markers for immune function/disease susceptibility.

Identification and Characterisation of New Mutations

Novel mutations in CD45 may be identified by scanning CD45 genomic sequence for genetic variation. The process of scanning CD45 genomic DNA for the presence of polymorphic variants may be accomplished using any of the techniques known in the art (see review by Schafer and Hawkins, Nature Biotechnology, Vol 16, pp 33-39 (1998)). Preferred techniques are listed below:

(a) DNA sequencing: Heterozygous changes appear as two bases at a single position in the sequence. Homozygous variants are found by comparison to a control (i.e. wild-type) sequence.

(b) Heteroduplex analysis: this technique is based on the fact that heteroduplexes exhibit a reduced mobility in non-denaturing polyacrylamide gels compared to homoduplexes. The region to be tested (advantageously around 200 bp) is amplified, denatured and re-natured to itself or control “wild-type” DNA and the duplexes resolved on a non-denaturing gel. The same region of DNA is compared between individuals and differential mobilities indicate sequence differences.

(c) Single-strand conformation polymorphism analysis (SSCP or SSCA): single stranded DNA folds up to form complex structures that are stabilized by weak intramolecular bonds. The electrophoretic mobilities of these structures on non-denaturing polyacrylamide gels is dependent upon chain length and conformation. Typically, PCR amplification products from the region to be tested are heat denatured and rapidly cooled to impede reassociation of complementary strands. The products are then resolved on a non-denaturing gel. The same region of DNA is compared between individuals and differential mobilities indicate sequence differences that exist between the individuals in this region.

(d) Chemical cleavage of mismatches (CCM): a radiolabelled probe is hybridised to the test DNA and mismatches detected by a series of chemical reactions that cleave one strand of the DNA at the site of the mismatch. This sensitive method can be applied to kilobase-length fragments.

(e) Enzymatic cleavage of mismatches: technique similar to CCM, except that the cleavage is performed using an enzyme (e.g. T4 endonuclease VII).

(f) Mass spectrometry: Matrix-assisted laser desorption/ionization time-of-flight mass spectrometry (MALDI-TOF-MS) may be used to compare DNA fragments by sensitive mass determination.

(g) Southern blotting: a labelled probe consisting of a fragment of the linkage region is hybridised to nylon membranes containing genomic DNA from patients and normal controls digested with different restriction enzymes. Large differences in the sizes of the restriction fragments hybridizing with the probe between patients and controls may indicate the presence of a restriction fragment length polymorphism.

(h) Denaturing high performance liquid chromatography (DHPLC): a PCR product is amplified corresponding to the region to be analysed for the presence of mutations. Heteroduplex formation is then analysed through hybridisation following heating and cooling of the PCR products.

The above-listed techniques may be employed to scan CD45 genomic DNA from human subjects in order to identify novel polymorphic variants.

In addition, a significant amount of information regarding known polymorphic variants is to be found in publicly accessible databases such as, for example, the human SNP database accessible via the Website of the Whitehead Institute, Cambridge, Mass., USA. Thus, it is also contemplated to scan/search these sources using in silico techniques to identify novel mutations/polymorphic variants in CD45 which may be used as the basis of genetic screens.

“New” CD45 mutations and variants may be characterised as Group I or Group II on the basis of their effect on (or association with) CD45 mRNA splicing and CD45 protein isoform expression. Such effects/associations may be investigated by analysing patterns of CD45 mRNA expression in individuals of know genotype, for example using RT-PCR or Northern blotting and/or analysing patterns of CD45 protein isoform expression on T lymphocytes in individuals of known genotype, for example using FACS analysis as described in the accompanying Examples.

Screens Based on Analysis of CD45 mRNA Expression and CD45 Protein Isoform Expression

The invention also relates to screens based on evaluation of the altered patterns of CD45 mRNA or protein isoform expression associated with carriage of CD45 Group I or Group II mutations. These screens are analogous to the genetic screens based on genotyping of CD45 mutations. In essence, all three screening methodologies ultimately provide an indication of carrier status for CD45 mutations, the only differences being whether the screen is carried out at the genomic level or at the level of mRNA or protein expression. Hence, all of the preferred clinical/diagnostic/prognostic uses described for the genetic screens are equally applicable to the screens based on altered mRNA or protein expression.

Accordingly, the invention provides a method of screening a human subject for susceptibility to viral infection and/or pre-disposition to developing severe disease following viral infection which comprises evaluating the pattern of CD45 mRNA expression in the subject, wherein the presence of an abnormal pattern of CD45 mRNA expression characterised by reduced splicing out of exon 4 of the CD45 mRNA and a quantitative decrease in amount of CD45R0 transcript is taken as an indication that the subject is susceptible to viral infection and/or pre-disposed to developing severe disease following viral infection.

In one embodiment the abnormal pattern of CD45 mRNA expression is that associated with the presence of a 77G mutant allele of the gene encoding CD45, the presence of this abnormal expression pattern being taken as an indication that the subject is more susceptible to viral infection and/or more pre-disposed to developing severe disease following viral infection, as compared to subjects who do not carry a 77G mutation.

In a further embodiment the abnormal pattern of CD45 mRNA expression is that associated with the presence of a 59A mutant allele of the gene encoding CD45, the presence of this abnormal expression pattern being taken as an indication that the subject is more susceptible to viral infection and/or more pre-disposed to developing severe disease following viral infection, as compared to subjects who do not carry a 59A mutation.

The term “abnormal pattern of CD45 mRNA expression associated with the presence of a 77G mutant allele of the gene encoding CD45“ refers to the variant CD45 splicing phenotype described by Thude et al., Eur J Immunol, 1995, Vol: 25(7), 2101-6 and shown to be associated with heterozygosity for the C77G mutation. Individuals homozygous for the C77G mutation are expected to show an exaggeration of the mRNA expression pattern observed in heterozygotes.

The term “abnormal pattern of CD45 mRNA expression associated with the presence of a 59A mutant allele of the gene encoding CD45” refers to the variant CD45 splicing phenotype described by Jacobsen, M. et al., Immunogenetics, 2002, Vol: 54(3), 158-63.

In a further aspect the invention provides a method of screening a human subject for susceptibility to viral infection and/or pre-disposition to developing severe disease following viral infection which comprises evaluating the pattern of CD45 mRNA expression in the subject, wherein the presence of an abnormal pattern of CD45 mRNA expression characterised by a quantitative increase in the level of expression of the CD45R0 transcript is taken as an indication that the subject is not susceptible to viral infection and/or is pre-disposed to developing less severe disease following viral infection.

In one embodiment the abnormal pattern of CD45 mRNA expression is that associated with the presence of a 138G mutant allele of the gene encoding CD45, wherein detection of the abnormal pattern of CD45 mRNA expression is taken as an indication that the subject is less susceptible to viral infection and/or pre-disposed to developing less severe disease following viral infection, as compared to subjects who do not carry a 138G mutant allele.

The invention also relates to a method of screening a human subject for an altered immune response capability, which method comprises evaluating the pattern of CD45 mRNA expression in said individual. The presence of an abnormal pattern of CD45 mRNA expression characterised by a quantitative increase in the level of expression of the CD45R0 transcript is taken as an indication that the subject has an altered immune response capability.

Again, in one embodiment the abnormal pattern of CD45 mRNA expression is that associated with the presence of a 138G mutant allele of the gene encoding CD45. Positive detection of the abnormal pattern of CD45 mRNA expression is taken as an indication that the subject has an altered immune response capability, as compared to subjects who do not carry a 138G mutant allele.

In a specific embodiment of this method the altered immune response capability is manifest as reduced susceptibility of a human subject to autoimmune disease. Detection of the abnormal pattern of CD45 mRNA expression (associated with carriage of a Group II allele, e.g. 138G) is taken as an indication that the subject has an altered immune response capability and therefore has reduced susceptibility to autoimmune disease, as compared to subjects who do not exhibit the abnormal pattern of CD45 mRNA expression.

The “abnormal pattern of CD45 mRNA expression associated with the presence of a 138G mutant allele of the gene encoding CD45” refers to the variant pattern of CD45 mRNA expression described by Stanton et al., PNAS, 2003, Vol 100(10), 5997-6002, the contents of which are incorporated herein by reference.

The screens based on analysis of patterns of CD45 mRNA expression are preferably carried out in vitro, for example by analysis of preparations of total or mRNA isolated from a tissue or cell type which expresses CD45 (e.g. peripheral blood lymphocytes). Suitable RNA analysis techniques which may be used to determine the pattern of CD45 mRNA expression in accordance with the invention include, but are not limited to, RT-PCR, NASBA, Northern blotting and RNAse protection assays, starting from a sample of total or mRNA prepared from a tissue which expresses CD45 (e.g. PBLs). It is most preferred to use a technique which permits quantitative analysis of mRNA expression, an in particular a technique that allows quantitation of the levels of expression of at least CD45R0 and CD45RA transcripts, and most preferably which allows quantitation of the levels of expression of all CD45 splice variants.

The invention also relates to analogous screening methods based on analysis of CD45 protein isoform expression.

Accordingly, the invention provides a method of screening a human subject for susceptibility to viral infection and/or pre-disposition to developing severe disease following viral infection which comprises evaluating the pattern of CD45 protein expression in the subject, wherein the presence of an abnormal pattern of CD45 protein expression characterised as an increased proportion of cells expressing CD45RA and a reduced proportion of single positive CD45RO+ T cells is taken as an indication that the subject is susceptible to viral infection and/or pre-disposed to developing severe disease following viral infection.

In one embodiment the abnormal pattern of CD45 protein expression is that associated with the presence of a 77G mutant allele of the gene encoding CD45. The presence of the abnormal pattern of CD45 protein expression is taken as an indication that the subject is more susceptible to viral infection and/or more pre-disposed to developing severe disease following viral infection, as compared to subjects who do not carry a 77G mutant allele.

In another embodiment the abnormal pattern of CD45 protein expression is that associated with the presence of a 54G mutant allele of the gene encoding CD45. The presence of the abnormal pattern of CD45 protein expression is taken as an indication that the subject is more susceptible to viral infection and/or more pre-disposed to developing severe disease following viral infection, as compared to subjects who do not carry a 54G mutant allele.

The invention also provides method of screening a human subject for susceptibility to viral infection and/or pre-disposition to developing severe disease following viral infection which comprises evaluating the pattern of CD45 protein expression in the subject, wherein the presence of an abnormal pattern of CD45 protein expression characterised by an increase in the proportion of T lymphocytes expressing the CD45R0 isoform but not CD45RA is taken as an indication that the subject is less susceptible to viral infection and/or is pre-disposed to developing less severe disease following viral infection.

In one embodiment the abnormal pattern of CD45 protein expression is that associated with the presence of a 138G mutant allele of the gene encoding CD45. The presence of the abnormal pattern of CD45 protein expression is taken as an indication that the subject is less susceptible to viral infection and/or pre-disposed to developing less severe disease following viral infection, as compared to subjects who do not carry a 138G mutant allele.

The invention still further relates to a method of screening a human subject for an altered immune response capability, which method comprises evaluating the pattern of CD45 protein expression in said individual, wherein the presence of an abnormal pattern of CD45 protein expression characterized as an increased in the proportion of T lymphocytes expressing the CD45R0 isoform but not CD45RA is taken as an indication that the individual has an altered immune response capability as compared to individuals that do not carry said mutation.

Again, in one embodiment the abnormal pattern of CD45 protein expression is that associated with the presence of a 138G mutant allele of the gene encoding CD45, wherein detection of the abnormal pattern of CD45 protein expression is taken as an indication that the subject has an altered immune response capability, as compared to subjects who do not carry a 138G mutant allele.

In a specific embodiment of this method the altered immune response capability is manifest as reduced susceptibility of a human subject to autoimmune disease. Detection of the abnormal pattern of CD45 protein expression (associated with carriage of a Group II allele, e.g. 138G) is taken as an indication that the subject has an altered immune response capability and therefore has reduced susceptibility to autoimmune disease, as compared to subjects who do not exhibit the abnormal pattern of CD45 protein expression.

The term “pattern of CD45 protein expression associated with the presence of a 77G mutant allele of the gene encoding CD45” refers to the variant pattern of expression of CD45 protein isoforms on peripheral T cells shown to be associated with heterozygosity for the C77G mutation, as described by Thude et al., Eur J Immunol, 1995, Vol: 25(7), 2101-6, the contents of which are incorporated herein by reference. The normal pattern of CD45 protein expression is characterised by loss of expression of the CD45RA isoform and gain in expression of CD45R0 after T cell activation. Individuals heterozygous for C77G are characterised continuous expression of the CD45RA isoform on activated and memory T cells, i.e. the T cells remain CD45RA/RO double positive after activation. Individuals homozygous for the C77G mutation are expected to show very little expression of CD45R0 at the cell surface.

The term “pattern of CD45 protein expression associated with the presence of a 59A mutant allele of the gene encoding CD45” refers to the variant pattern of expression of CD45 protein isoforms on peripheral T cells described by Jacobsen, M. et al., 2002, Immunogenetics, Vol: 54(3), 158-163, the contents of which are incorporated herein by reference.

The term “pattern of CD45 protein expression associated with the presence of a 138G mutant allele of the gene encoding CD45” refers to the variant pattern of expression of CD45 protein isoforms on peripheral T cells described by Stanton et al., PNAS, 2003, Vol 100(10), 5997-6002.

Analysis of the CD45 protein isoform expression pattern on peripheral T cells is preferably carried out using flow cytometry, as described in the accompanying example. Individuals heterozygous for C77G are characterised by the absence of a CD45RA negative population of leucocytes. Further suitable techniques which may be used to assess the pattern of expression of CD45 isoforms include, but are not limited to, immunoprecipitation and Western blotting.

In a further aspect, the invention provides a method of screening an individual for an altered immune response, which method comprises evaluating the pattern of CD45 protein expression in said individual, wherein the presence of an abnormal pattern of CD45 protein expression associated with the presence of a 138G mutant allele of the gene encoding CD45 is taken as an indication that the individual has an altered immune response profile as compared to individuals that do not carry said mutation.

The term “pattern of CD45 protein expression associated with the presence of a A138G mutant allele of the gene encoding CD45” refers to the variant pattern of expression of CD45 protein isoforms on peripheral T cells shown to be associated with heterozygosity for the A138G mutation. The normal pattern of CD45 protein expression is characterised by loss of expression of the CD45RA isoform and gain in expression of CD45R0 after T cell activation.

The screens based on analysis of the CD45 protein isoform expression pattern on peripheral T cells are preferably carried out in vitro on samples removed from the subject under test. Analysis of the CD45 protein isoform expression pattern on peripheral T cells is preferably carried out using flow cytometry, as described in the accompanying examples, but other techniques for the analysis of protein expression may be used. Further suitable techniques which may be used to assess the pattern of expression of CD45 isoforms include immunoprecipitation and Western blotting.

Generally it is preferred to analyse CD45 protein isoform expression using a technique which permits quantitation of the levels of expression of at least the CD45R0 and CD45RA isoforms, and more preferably using a technique that allows quantitation of the levels of expression of all CD45 protein isoforms.

All of the screening methods of the invention may be used to identify human subjects who are susceptible or pre-disposed to viral infection by virtue of their genetic make-up. This may allow intervention with preventative therapies aimed at boosting immune function. Screening for increased susceptibility to viral infections and/or for risk of developing more severe virus-induced disease would be important for individuals at increased risk of life threatening virus infections. These may include, for example, gay men and intravenous drug users or medical personnel working in renal dialysis units. “At risk” individuals may be counselled or excluded from high risk situations and measures may be taken to ensure that vaccination results in protective antibody titres in these individuals where a vaccine is available. Screening may also be useful for predicting whether individuals with chronic viral infection, such as for example Hepatitis B or C, are likely to be refractory to expensive immunotherapy.

Kits

The invention also relates to kits for use in carrying out the methods of the invention. In particular embodiments the invention provides:

(1) A kit for use in genotyping individuals for the A138G polymorphism by amplification refractory mutation system (ARMS) PCR, the kit comprising the following oligonucleotide primers:

| | |
|---|---|
| 5′-GGAGAAGTGCTTGAAGATT-3′, | (SEQ ID NO:1) |
| 5′-CGTATCAGTCTGGACTCCA-3′, | (SEQ ID NO:2) |
| and | |
| 5′-CGTATCAGTCTGGACTCCG-3′. | (SEQ ID NO:3) |

(2) A kit for use in genotyping individuals for the C77G polymorphism by PCR RFLP, the kit comprising the following oligonucleotide primers:
5′-GACTACAGCAAAGATGCCCAGTG-3′ (SEQ ID NO:4) and
5′-GGGATACTTGGGTGGAAGTA-3′ (SEQ ID NO:5), optionally with a supply of the restriction enzyme Msp I.

(3) A kit for use in genotyping individuals for the C77G polymorphism by amplification refractory mutation system (ARMS) PCR, the kit comprising the following oligonucleotide primers:

| | |
|---|---|
| 5′-CATATTTATTTTGTCCTTCTCCCA-3′, | (SEQ ID NO:6) |
| 5′-GAAAGTTTCCACCAACGG-3′ | (SEQ ID NO:7) |
| and | |
| 5′-GAAAGTTTCCACGAACGC-3′. | (SEQ ID NO:8) |

(4) A kit for use in genotyping individuals for the A54G polymorphism and/or the C77G polymorphism by DHPLC, the kit comprising the following oligonucleotide primers:

| | |
|---|---|
| 5′-CATATTTATTTTGTCCTTCTCCCA-3′ | (SEQ ID NO:6) |
| and | |
| 5′-GTGCAGAAATGCAGGAAAT-3′. | (SEQ ID NO:9) |

(5) A kit for use in genotyping individuals for the A138G polymorphism by DHPLC, the kit comprising the following oligonucleotide primers:

| | |
|---|---|
| 5′-GGAGAAGTGCTTGAAGATT-3′ | (SEQ ID NO:1) |
| and | |
| 5′-GTGCCAGATATTATTTGTAGG-3′. | (SEQ ID NO:10) |

(6) A kit for use in genotyping individuals for the A138G polymorphism by amplification refractory mutation system (ARMS) PCR, the kit comprising the following oligonucleotide primers:

| | |
|---|---|
| 5′-GCAGAAGTGCTTGAAGATT-3′ | (SEQ ID NO:19) |
| 5′-GCATAGTCAGACCTGAGCT-3′ | (SEQ ID NO:20) |
| and | |
| 5′-GCATAGTCAGACCTGAGCC-3′. | (SEQ ID NO:21) |

(7) A kit for use in genotying the A138G mutation by Sequenom Mass-Array® MALDI-TOF primer extension assay, the kit comprising the following oligonucleotide primers:

(SEQ ID NO:25)

P1:
5′-ACGTTGGATGACCTCCAACACCACCATCAC-3′,

(SEQ ID NO:26)

P2:
5′-ACGTTGGATGGAAGACACTACTAGAGCAGC-3′
and

(SEQ ID NO:27)

extension P:
5′-ACACCACCATCACAGCGAAC-3′.

(8) A kit for use in genotying the C77G mutation by Sequenom Mass-Array® MALDI-TOF primer extension assay, the kit comprising the following oligonucleotide primers:

| | |
|---|---|
| P1: | |
| 5′-ACGTTGGATGCTTTCAAGTGACCCCTTACC-3′, | (SEQ ID NO:28) |
| P2: | |
| 5′-ACGTTGGATGGTTGTGGTCTCTGAGAAGTC-3′ | (SEQ ID NO:29) |
| and extension | |
| P: | |
| 5′-CCACTGCATTCTCACC-3′. | (SEQ ID NO:30) |

Oligonucleotide primers may be supplied in the kits ready for use, as concentrates requiring dilution before use, or in a lyophilised or dried form requiring reconstitution prior to use. If required, the kits may further include a supply of a suitable diluent for dilution or reconstitution of the primers. Optionally, the kits may further comprise supplies of reagents, buffers, enzymes etc for use in carrying out PCR amplification. The preferred features of such reagents are described in the Materials and Methods sections of the accompanying Examples.

Transgenic Mammals

The inventors have shown experimentally in CD45^{RABC/+} and CD45^{RO/+} transgenic mice that altering the combination of CD45 isoforms expressed can dramatically affect immune function and also disease severity in an experimental model of autoimmune disease.

Accordingly, the invention also encompasses non-human transgenic mammals having the genotype CD45^{RABC/+} and non-human transgenic mammals having the genotype CD45R0^{/+}.

In a preferred embodiment the CD45^{RABC/+} transgenic mammals are heterozygotes expressing a single copy of a fixed non-splicing single CD45 transgene which encodes only the CD45 RABC isoform on a heterozygous knockout/wild-type genetic background.

In a preferred embodiment the CD45^{RO/+} transgenic mammals are heterozygotes expressing a single copy of a fixed non-splicing single CD45 transgene which encodes only the CD45 RO isoform on a heterozygous knockout/wild-type genetic background.

The inventors have found that transgenic mammals according to the invention, expressing one allele encoding a fixed CD45 isoform and one normally splicing allele, provide more accurate models of the altered immune response capabilities observed in human individuals carrying group I or group II CD45 mutations than transgenic animal models expressing single CD45 isoforms. The inventors conclude that the relative levels of expression of the different CD45 isoforms are important in determining immune function, rather than the absolute presence or absence of particular isoforms.

Methods for generating transgenic mammals according to the invention are generally well known in the art (see Gene Targeting: A Practical Approach, Joyner, ed., Oxford University Press, 2000). In brief, transgenic mammals expressing single CD45 isoforms can be created by introducing a transgene construct which encodes only the single CD45 isoform of choice onto a CD45−/− genetic background. By way of example, construction of transgenic mice expressing single CD45R0 and CD45RABC isoforms on an exon 9-targeted CD45−/− genetic background is described in detail by Tchilian et al. International Immunology, Vol. 16, pp.1323-1332, 2004, the contents of which are incorporated herein by reference. Heterozygotes carrying one CD45 allele which encodes a single CD45 isoform and one normally splicing CD45 allele can then be constructed by crossing a single isoform transgenic animal with an animal homozygous for the normally splicing allele according to standard breeding techniques, as illustrated in the accompanying examples.

The CD45^{RABC/+} and CD45^{RO/+} transgenic mammals provided by the invention provide useful models of the altered immune function associated with variation in the levels of expression of different CD45 isoforms. As illustrated in the accompanying examples, both CD45^{RABC/+} and CD45^{RO/+} transgenic mice show altered kinetics and magnitude of proliferative responses. The outcome of these changes is an altered threshold for TCR signaling reflected in the rapid proliferative responses of CD45^{RABC/+} or CD45R0^{/+} cells. As a result of the more vigorous TCR response, more TNFα and IFNγ is produced by CD45^{RABC/+} or CD45R0^{/+} cells.

The most striking effects of altered CD45 isoforms expression in CD45^{RABC/+} or CD45R0^{/+} transgenic mammals were observed in an experimental model of autoimmune disease—experimental autoimmune encephalomyelitis (EAE). Both CD45^{RABC/+} and CD45R0^{/+} mice showed more rapid onset and increased severity of disease than CD45^{+/+} mice, with the most severe disease occurring in CD45R0^{/+} mice. Thus, the inventors have shown experimentally that altering the combination of CD45 isoforms dramatically affects immune function and disease severity in an autoimmune model. The mechanism is an altered threshold for TCR signaling and altered cytokine production and response. This in turn indicates that manipulating the patterns of CD45 expression or signaling pathways it modulates will be a useful therapeutic strategy.

CD45^{RABC/+} and CD45R0^{/+} transgenic mammals according to the invention find specific utility as research tools for the study of the effect of CD45 isoform expression on immune function. More specifically CD45^{RABC/+} or CD45R0^{/+} transgenic mammals can be used to study, respectively, the mechanisms underlying altered disease susceptibility in humans carrying Group I or Group II CD45 mutations. In a particularly useful embodiment transgenic mammals (e.g. transgenic mice) according to the invention can be challenged with Myelin Oligodendrocyte Glycoprotein (MOG) peptide in order to induce EAE and then used to study the mechanisms underlying the relationship between altered CD45 isoform expression and severity of autoimmune disease in this model.

The transgenic mammals according to the invention (including those subsequently challenged to induce EAE), and/or cell lines or tissues derived therefrom, also provide useful model systems for testing the efficacy, safety, toxicity and specificity of potential therapeutic agents targeted at altering the function or expression of particular CD45 isoforms, or combinations thereof.

As aforesaid, the inventors' observation that altering the combination of CD45 isoforms expressed in transgenic mice dramatically affects immune function and disease severity in an autoimmune model indicates that manipulating the patterns of CD45 expression or signaling pathways that it modulates will be a useful therapeutic strategy. Furthermore, the inventors' observations using transgenic mice expressing single CD45RABC or CD45RO transgenes on a CD45KO background (obtained as described in Tchilian et al, Int Immunol, 2004, 16: 1323-1332) indicate that alterations in the level of CD45 expressed also affect immune responses. Therefore therapeutic strategies modulating either the absolute level of CD45 expression, the combination of isoforms expressed or relative levels of isoform expression may be useful in autoimmune and infectious diseases.

Therapeutic interventions targeted at specific CD45 isoforms may be useful in the treatment of diseases associated with altered patterns and/or levels of CD45 isoform expression. CD45^{RABC/+} and CD45R0^{/+} transgenic mammals provide useful model systems in which to test the potential efficacy of therapeutic agents which target specific CD45 isoforms, since they provide more accurate models of immune function in humans carrying Group I or Group II CD45 mutations than single isoform transgenic animals.

In the context of this application the terms “therapy” and “therapeutic treatment” refer to any intervention aimed at alleviating, reducing, preventing, lessening the severity of or eliminating one or more symptoms of a disease or condition, including prophylactic interventions in substantially asymptomatic individuals.

Therapeutic strategies for targeting single CD45 isoforms may be based on any substance which is a specific antagonist of a single CD45 protein isoform or a subset of CD45 isoforms. In this context the term “specific antagonist” includes antagonist substances that are specific for a single CD45 isoform and even substances which are antagonists of a subset of CD45 isoforms, for example the subset of isoforms which contain alternative exon A, but excludes substances which are antagonists for all CD45 isoforms.

Specific CD45 antagonists may include antibodies capable of binding with immunological specificity to single CD45 isoforms or a subset of CD45 isoforms, also natural or synthetic antibody fragments which retain specificity of binding to the specific CD45 isoform including, inter alia, F(ab′)₂ fragments, Fv fragments, also single chain antibodies (scabs), single chain Fv fragments (scFv), single domain antibodies (dAb), camelids, nanobodies, or other antibody mimetics. Monoclonal antibodies specific for CD45R0, CD45RA and CD45RB isoforms have been described in the art. For description of anti-CD45R0 antibodies see Smith et al. Immunology, 58, pp. 63-70, 1986. For description of anti-CD45RA antibodies see Knapp W, Dorken B and E P Rieber, et al, eds 1989. Leucocyte Typing IV: White Cell Differentiation Antigens, Oxford University Press, New York, Barclay A N et al. 1993. The Lueccocyte Antigen Facts Book, MHC section, Academic Press, Inc., San Diego, Calif. p202, Cobbold S, Hale G, Waldemann H. Non-lineage, LFA-1 family and leucocyte common antigens: new and previously defined clusters. In McMichale A J, ed Leucocyte Typing III: White Cell Differentiation Antigens. Oxford: Oxford University Press; 1987:788-802. For description of anti-CD45RB antibodies see Basadonna et al., PNAS, 95, pp 3821-3826, 1998.

In addition to the foregoing, natural or synthetic small molecule inhibitors of single CD45 isoforms, or subsets of isoforms, are not excluded.

Therapeutic strategies may also be based on targeted down-regulation of expression of mRNAs encoding specific CD45 isoforms or subsets of isoforms. In this case, therapeutic intervention would occur at the level of mRNA expression rather than activity of the encoded protein.

Various generic approaches to targeted down-regulation of mRNA expression are known in the art, one of the most promising of which is based on RNA interference (RNAi), particularly using small interfering or “siRNAs”.

RNA interference or “RNAi” is a process of sequence-specific down-regulation of gene expression (also referred to as “gene silencing” or “RNA-mediated gene silencing”) initiated by double-stranded RNA (dsRNA) that is complementary in sequence to a region of the target gene to be down-regulated (Fire, A. Trends Genet. Vol. 15, 358-363, 1999; Sharp, P. A. Genes Dev. Vol. 15, 485-490, 2001).

Over the last few years, down-regulation of target genes in multicellular organisms by means of RNA interference (RNAi) has become a well established technique. In general, RNAi comprises contacting the organism with a double-stranded RNA fragment (generally either as two annealed complementary single-strands of RNA or as a hairpin construct) having a sequence that corresponds to at least part of a gene to be down-regulated. Reference may be made to International application WO 99/32619 (Carnegie Institute of Washington), International application WO 99/53050 (Benitec), and to Fire et al., Nature, Vol. 391, pp.806-811, February 1998 for general description of the RNAi technique.

Elbashir et al. (Nature, 411, 494-498, 2001) have demonstrated effective RNAi-mediated gene silencing in mammalian cells using dsRNA fragments of 21 nucleotides in length (also termed small interfering RNAs or siRNAs). These short siRNAs demonstrate effective and specific gene silencing, whilst avoiding the interferon-mediated non-specific reduction in gene expression which has been observed with the use of dsRNAs greater than 30 bp in length in mammalian cells (Stark G. R. et al., Ann Rev Biochem. 1998, 67: 227-264; Manche, L et al., Mol Cell Biol., 1992, 12: 5238-5248). Thus, RNAi can be used for specific down-regulation or gene silencing of target genes in mammalian cells.

siRNA reagents capable of targeting specific CD45 isoforms or subsets of isoforms can be designed using techniques generally well known in the art.

Further therapeutic interventions may be based on the use of targeted gene therapy to alter the balance of CD45 isoform expression in particular cells or tissues.

The inventors' studies in transgenic mice, as described in the accompanying examples, indicate that reducing the total level of CD45 expression leads to reduced immune responses and increased apoptosis of leucocytes. Such reduction of the immune response may be beneficial in the treatment or prevention of autoimmune disease conditions and/or lymphoproliferative diseases. The invention therefore contemplates a method of treating and/or preventing autoimmune or lymphoproliferative disease in a human patient comprising administering to a patient in need thereof an effective amount of an antagonist of one or more isoforms of CD45.

In specific embodiments the antagonist may be an antibody, antibody fragment or synthetic antibody mimetic as described above or an siRNA reagent capable of specifically down-regulating expression of one or more isoforms of CD45, also as described above. The antagonist may antagonise all CD45 isoforms, a single isoform or a subset of isoforms. Exemplary autoimmune diseases include multiple sclerosis, Grave's disease, Hashimoto's thyroiditis, type I diabetes, rheumatoid arthritis and systemic lupus erythmatosus.

The inventors' have also observed that an excess of the CD45RABC isoform produces an immune response which is generally detrimental, such that correction or normalisation of the level of expression of this specific isoform is beneficial in the treatment or prevention of autoimmune disease, particularly MS. In particular, the inventors' have observed that normalisation of CD45RABC expression results in less severe disease in a mouse EAE disease model. Therefore, the invention also contemplates a method of treating or preventing autoimmune disease in a human patient comprising administering to a patient in need thereof an effective amount of an antagonist of the CD45RABC isoform.

In a preferred embodiment the antagonist of CD45RABC will be a specific CD45RABC antagonist. Exemplary autoimmune diseases again include multiple sclerosis, Grave's disease, Hashimoto's thyroiditis, type I diabetes, rheumatoid arthritis and systemic lupus erythmatosus.

Increased expression of the CD45RO isoform is predicted to be of beneficial effect in certain infectious diseases (in particular viral infections) and autoimmune disease, due to increased production of IFN gamma and TNF. Therefore, the invention still further contemplates a method of treating or preventing infectious diseases, and in particular viral infections, or autoimmune disease in a human patient by administering to a patient in need thereof an effective amount of an agent which up-regulates the expression and/or activity of the CD45RO isoform. A specific embodiment may relate to the use of targeted gene therapy to express a transgene encoding the CD45RO isoform in at least one cell type or tissue (e.g. lymph node T cells). Suitable expression vectors etc. and transformation techniques for targeting expression of a particular transgene of interest to a particular cell type or tissue are generally known in the art.

The invention will be further understood with reference to the following, non-limiting, Experimental Examples.

### EXAMPLES

### Example 1

### Association Between C77G and HIV Infection

Genomic DNA samples and cryopreserved PBMC were obtained from 182 HIV-1 infected patients enrolled at the St Stephen's Clinic, Chelsea and Westminster Hospital, as a part of a functional immunological study. An additional 15 DNA samples from individuals identified as HIV-1-infected at seroconversion, were supplied by Dr P. Borrow. Ethical approval was obtained and the patients gave written consent. The control group consisted of 236 healthy volunteer blood donors, obtained through the local blood bank of the UK National Blood Transfusion Service.

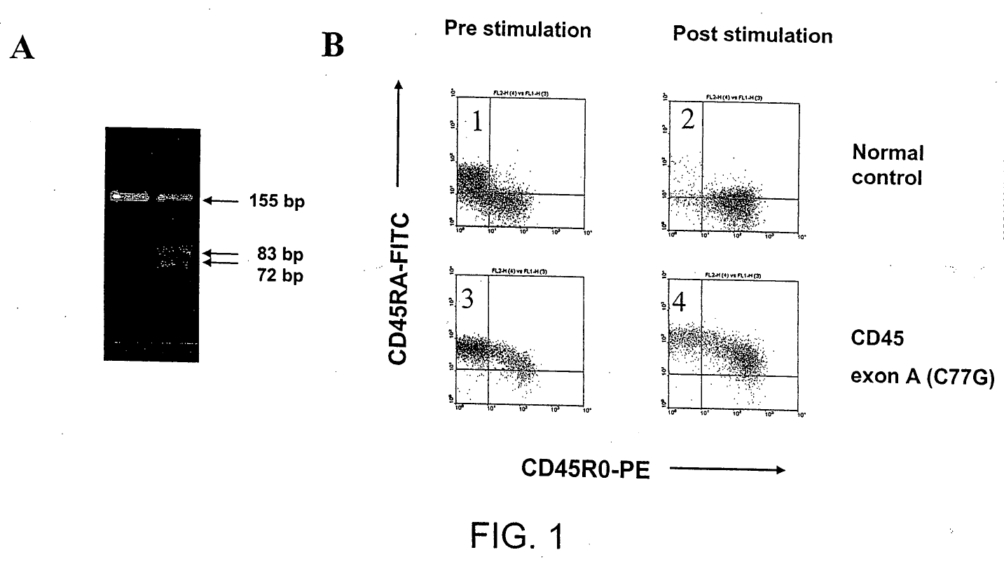

The detection of exon A (C77G) was performed on genomic DNA amplified by PCR using forward (5′-GACTACAGCAAAGATGCCCAGTG-3′ (SEQ ID NO:4)) and reverse primers (5′-GGGATACTTGGGTGGAAGTA-3′ (SEQ ID NO:5)). The C77G transition introduces a new restriction site for Msp I, which cleaves the mutant PCR product into two fragments of 72 and 83 bp. The presence of an undigested band of 155 bp indicates the presence of the wild type allele (illustrated in FIG. 1A).

The presence of the CD45 exon A mutant allele was confirmed by sequencing and flow cytometric analysis on C77G positive samples. PBMC were stimulated with PHA and on days 0 and day 10 stained with isoform specific CD45R0-PE and CD45RA-FITC antibody conjugates (obtained from Dako and Sigma, respectively) together with CD3-APC antibodies (obtained from Pharmingen). Analysis was performed on gated CD3+ T cells. The normal pattern of CD45 splicing is characterised by loss of CD45RA and gain in expression of CD45R0 associated with the activated/memory function (A and B, FIG. 1). Variant CD45 splicing can be identified by the absence of the single CD45R0+ population and even after 10 days of stimulation the T cells remain CD45RA/RO double positive (C and D, FIG. 1).

Using PCR and Msp I digestion analysis 11 individuals with the exon A (C77G) mutation were identified out of 197 HIV-1 patients (allele frequency 5.6%) and 4 out of 236 healthy donors (allele frequency 1.7%). The presence of the C77G mutation in these individuals was confirmed by flow cytometric analysis of CD45 protein expression. Using two-tailed Fisher's exact test to test for the association between C77G mutation and HIV-1 infection, a statistically significant association was demonstrated (p=0.03).

The results of this study clearly indicate that exon A (C77G) transversion and abnormal CD45 splicing are associated with HIV-1 infection.

### Example 2

### Abnormal CD45 Splicing in Haemophagocytic Lymphohistiocytosis

Two patients with a similar defect in CD45 splicing associated with familial erythrophagocytic lymphohistiocytosis (Bujan, W., L. Schandene, A. Ferster, C. De Valck, M. Goldman, and E. Sariban. 1993. Lancet 342:1296) and haemophagocytic lymphohistiocytosis (Wagner, R., G. Morgan, and S. Strobel. 1995. Clin. Exp. Immunol. 99:216.) have been previously described. Haemophagocytic lymphohistiocytosis (HLH) is a rare disorder characterised by disregulated activation of T lymphocytes and macrophages (Arico, M., S. Imashuku, R. Clementi, S. Hibi, T. Teramura, C. Danesino, D. A. Haber, and K. E. Nichols. 2001. Blood 97:1131). HLH is genetically heterogenous with both familial and sporadic forms described (Janka, G. E. 1983. Eur. J. Pediatr. 140:221; Dreyer, Z. E., B. L. Dowell, H. Chen, E. Hawkins, and K. L. McClain. 1991. Am J Pediatr Hematol Oncol vol. 13:476; Dufourcq-Lagelouse, R., N. Jabado, F. Le Deist, J. L. Stephan, G. Souillet, M. Bruin, E. Vilmer, M. Schneider, G. Janka, A. Fischer, and G. de Saint Basile. 1999. Am. J. Hum. Genet. 64:172).

Because of the similarity of the abnormal CD45 splicing in the two previously described HLH patients, to variant CD45 splicing in apparently normal individuals, we investigated the association of the known C77G mutation and HLH syndrome.

Materials and Methods

Materials

Fresh blood was obtained from the previously described family W. (Wagner, R., G. Morgan, and S. Strobel. 1995. Clin. Exp. Immunol. 99:216) and family G. (with two children with HLH) from the Immunobiology Unit, Institute for Child Health, London, UK. PBMC were isolated by centrifugation on a Ficoll-Paque (Amersham Pharmacia Biotech, Buckinghamshire, UK) density gradient and genomic DNA was extracted by standard procedures (Sambrook, J., E. F. Fritsch, and T. Maniatis. 1989. Molecular Cloning: A Laboratory Manual. Cold Spring Harbour Laboratory Press). Genomic DNA samples from family R. (Bujan, W., L. Schandene, A. Ferster, C. De Valck, M. Goldman, and E. Sariban. 1993. Lancet 342:1296.) together with genomic DNA samples from 19 unrelated HLH patients were provided by the Universita di Pavia, Italy.

Genotyping for CD45 Exon A (C77G) Mutation

Genomic DNA was amplified by PCR using forward (5′-GACTACAGCAAAGATGCCCAGTG-3′ (SEQ ID NO:4)) and reverse (5′-GGGATACTTGGGTGGAAGTA-3′ (SEQ ID NO:5)) primers as previously described in Example 1. The C77G transversion introduces a new restriction site for MspI (Amersham Pharmacia Biotech), which produces two additional fragments of 72 bp and 83 bp upon digestion in the mutant allele. The PCR and digestion products were analysed on VisiGel Separation Matrix (Stratagene, La Jolla, Calif.).

Flow Cytometric Analysis

Flow cytometric analysis of CD45 variant splicing was performed as previously described (Tchilian, E. Z., D. L. Wallace, N. Imami, H. X. Liao, C. Burton, F. Gotch, J. Martinson, B. F. Haynes, and P. C. Beverley. 2001. J. Immunol. 166:6144.). Briefly, 2×10⁵ PBMC were stained with APC-conjugated CD3 (Pharmingen, SanDiego, Calif.) along with FITC-conjugated CD45RA (Sigma, St Louis, Mo.) and PE-conjugated CD45R0 (Dako, Glostrup, Denmark) mabs in a single step at 4° C. for 20 minutes and washed with PBS, containing 0.5% BSA. Isotype matched mAbs were used as controls. 10,000 events per sample were collected on FACSCalibur (Becton Dickenson, Mountain View, Calif.) and analysed with Cellquest software.

Results

CD45 Exon A (C77G) Mutation is the Cause of CD45 Abnormal Splicing in Two Families with HLH

Material was obtained from two patients with HLH, previously described as exhibiting CD45 abnormal splicing as characterised by the lack of the single CD45R0+ T cell population (Bujan, W., L. Schandene, A. Ferster, C. De Valck, M. Goldman, and E. Sariban. 1993. Lancet 342:1296; Wagner, R., G. Morgan, and S. Strobel. 1995. Clin. Exp. Immunol. 99:216). Subsequently a C77G mutation in exon A of CD45 has been shown to be responsible for the abnormal CD45 splicing in T lymphocytes (Thude, H., J. Hundrieser, K. Wonigeit, and R. Schwinzer. 1995. Eur. J. Immunol. 25:2101; Zilch, C. F., A. M. Walker, M. Timon, L. K. Goff, D. L. Wallace, and P. C. Beverley. 1998. Eur. J. Immunol. 28:22). We therefore genotyped these patients and members of their families for the presence of the CD45 exon A C77G mutation.

Patient W. was the third child of healthy unrelated British Caucasian parents (Wagner, R., G. Morgan, and S. Strobel. 1995. Clin. Exp. Immunol. 99:216.). He presented aged 3 mo with fever, diarrhoea, pallor, increasing irritability and marked cervical lymphoadenopathy and hepatosplenomegaly. Laboratory investigations revealed pancytopenia, coagulopathy and hypertryglyceridemia. The diagnosis of HLH was made from the bone marrow aspirate, which showed haemophagocytosis. There was a good response to initial treatment with dexamethasone and etoposide and he underwent allogeneic bone marrow transplantation from his HLA identical brother.

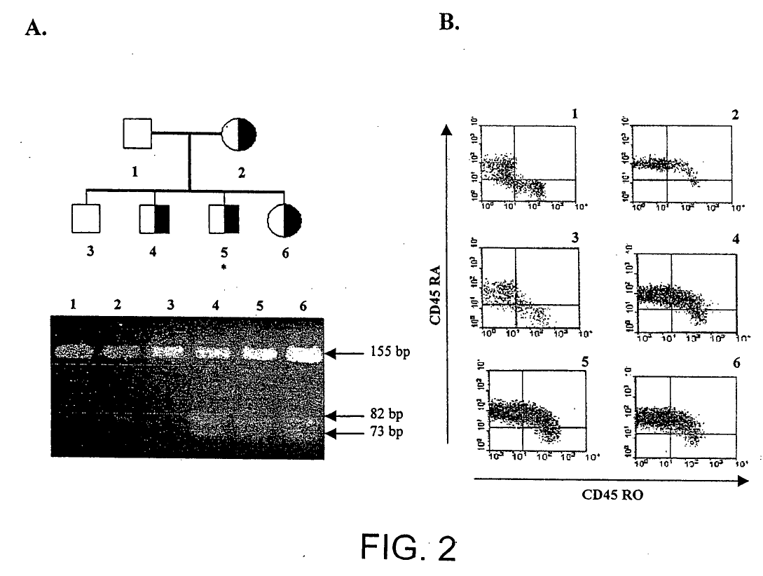

Using PCR and MspI restriction analysis we found that patient W. his mother and two siblings were heterozygous for the mutant C77G allele while the father and the oldest brother had wild type CD45 (FIG. 2A). These results were confirmed by flow cytometric analysis on PBMC from family W. (FIG. 2B). All of the family members genotyped as having the C77G mutation exhibit phenotypically abnormal CD45 splicing. (These results are in agreement with the initial report of family W. that at that time had only three children.)

Patient R. was a first child of consanguineous Belgian Caucasian parents (Bujan, W., L. Schandene, A. Ferster, C. De Valck, M. Goldman, and E. Sariban. 1993. Lancet 342:1296.). The patient presented at the age of 2 mo with fever, hepatosplenomegaly, neutropenia, thrombocytopenia, hypofibrinogenemia and hypertriglyceridemia. He responded to initial treatment with etoposide and underwent bone marrow transplantation from his haploidentical half-sibling and remained asymptomatic over 8 years later. Two older siblings from a previous marriage died during infancy of a histiocytic disorder. Genotyping for the C77G polymorphism revealed that the patient and his mother are heterozygotes while his father and grandmother (also the father's sister) carried wild type CD45 (data not shown). Taken together these results show that the CD45 exon A (C77G) mutation is the cause for the CD45 abnormal splicing in the two HLH patients.

Analysis of CD45 Exon A C77G Mutation in 21 HLH Patients

Since two families with HLH were identified with abnormal CD45 splicing and the C77G mutation we next investigated the pattern of CD45 expression in other HLH patients. Using PCR and MspI restriction analysis we genotyped 21 patients with HLH (including the two affected sibs from family G.) for the presence of CD45 exon A (C77G) mutation. We did not find the mutant C77G allele in any of these patients.

Although taken together the above results show a frequency of 1:10 in HLH type 2 (with identified mutations in the PRF1 gene), or 2:23 for HLH overall, the number of subjects included in the study was very small and it is therefore impossible to draw statistically significant conclusions. Extensive studies on the frequency of C77G have not been performed but we have shown the frequency of the C77G individuals to be about 1.76% in the UK (Tchilian, E. Z., D. L. Wallace, N. Imami, H. X. Liao, C. Burton, F. Gotch, J. Martinson, B. F. Haynes, and P. C. Beverley. 2001. J. Immunol. 166:6144.), while in Germany the frequency has been reported to be less then 1% and in North America to be higher (3.6 %) (Jacobsen, M., D. Schweer, A. Ziegler, R. Gaber, S. Schock, R. Schwinzer, K. Wonigeit, R. B. Lindert, O. Kantarci, J. Schaefer-Klein, H. I. Schipper, W. H. Oertel, F. Heidenreich, B. G. Weinshenker, N. Sommer, and B. Hemmer. 2000. Nat. Genet. 26:495.).

### Example 3

### Abnormal CD45 Splicing in a Patient with a Common Variable Immunodeficiency and a History of Prolonged Faecal Excretion of Poliovirus.

Common variable immunodeficiency (CVID) is an acquired primary antibody deficiency characterised by recurrent encapsulated bacterial infection and autoimmune disease. The underlying pathogenic defects are heterogeneous with at least four groups of patients being identified according to their ability to secrete immunoglobulin in vitro (Bryant A, Calver N C, Toubi E, Webster A D, Farrant J. Clin Immunol Immunopathol 1990; 56: 239-48), presence of granulomatous disease and autoimmune disease. In general, patients with CVID are not prone to viral infections though infection with enteroviruses may be a potential problem (Rudge P, Webster A D, Revesz T, et al. Brain 1996; 119: 1-15). In view of the possibility that abnormalities in CD45 splicing might contribute to impaired anti-viral responses we report here on a patient with CVID and a history of prolonged poliovirus excretion (Misbah S A, Lawrence P A, Kurtz J B, Chapel H M. Postgrad Med J 1991; 67: 301-303), who exhibited abnormal CD45 splicing caused by the C77G polymorphism.

Materials and Methods

Case History

The patient was a 49 year old Caucasian male with CVID who had prolonged faecal excretion of a non-vaccine strain type II poliovirus between January 1987 and July 1988. In view of his occupation as a nurse and the attendant occupational health implications of prolonged poliovirus excretion, his case history was previously reported (Misbah S A, Lawrence P A, Kurtz J B, Chapel H M. Postgrad Med J 1991; 67: 301-303). In brief CVID was diagnosed at the age of 18 years when he presented with hypogammaglobulinaemia (IgG 2.8 g/l (ref. range 8-16), IgA 0.48 g/l (ref. range 1.4-4.2), IgM undetectable (ref. range 0.5-2.0)) on a background of delayed puberty, intermittent diarrhoea and intestinal nodular lymphoid hyperplasia. He was lost to follow-up between 1972 and 1986. Although he did not suffer from recurrent infections, it was thought prudent to commence him on intramuscular immunoglobulin therapy in January 1987 because of his occupation as a nurse. He has been maintained on replacement immunoglobulin since, switching from intramuscular to subcutaneous immunoglobulin in September 1998. Trough serum IgG levels have varied between 4.4 to 6.1 g/l over the past 2 years. His clinical progress on immunoglobulin replacement has been excellent with only occasional episodes of diarrhoea.

Materials

Fresh EDTA blood was obtained from the patient (Misbah S A, Lawrence P A, Kurtz J B, Chapel H M. Postgrad Med J 1991; 67: 301-303) via the Department of Immunology, John Radcliffe Hospital, Oxford, UK. Genomic DNA was extracted by standard procedure (Sambrook J, E. F. Fritsch, and T. Maniatis. Molecular Cloning: A Laboratory Manual. Cold Spring Harbour Laboratory Press 1989) and monoclonal antibody staining performed as described below.

Genotyping for CD45 Exon A (C77G) Mutation

Genomic DNA was amplified by PCR using forward (5′-GACTACAGCAAAGATGCCCAGTG-3′ (SEQ ID NO:4)) and reverse (5′-GGGATACTTGGGTGGAAGTA-3′ (SEQ ID NO:5)) primers as described in Example 1 and PCR products carrying the C77G transversion identified by digestions with MspI, which produces two additional fragments of 72 bp and 83 bp upon digestion in the mutant allele.

Flow Cytometric Analysis

Flow cytometric analysis of CD45 variant splicing was adapted from the method previously described (Tchilian E Z, Wallace D L, Imami N, et al. J Immunol 2001; 166: 6144-8). Briefly, 50 μl of EDTA blood was stained with PerCP-conjugated CD3, FITC-conjugated CD45RA and PE-conjugated CD45R0 (Becton Dickinson, Oxford, UK) monoclonal antibodies for 15 minutes in the dark at room temperature. Red blood cells were lysed by addition of 1 ml of FACSlyse (Becton Dickinson) for 10 minutes. Lysed stained cells were washed twice with sheath fluid (Becton Dickinson) before being fixed in 0.4 ml of 1% paraformaldehyde and analysed on a FACScan flow cytometer (Becton Dickinson) using Cellquest software. 10,000 events per sample were collected and isotype matched mAbs were used as controls.

Results

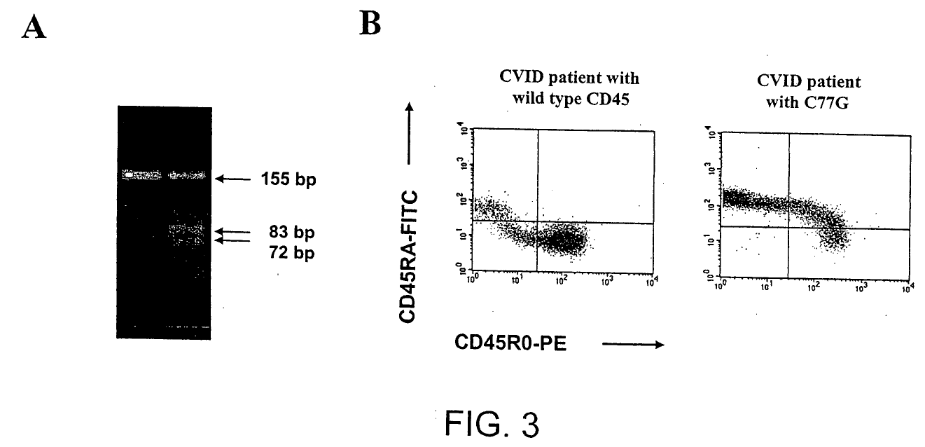

Using PCR and MspI restriction analysis we found that the patient was heterozygous for the mutant C77G allele (FIG. 3A). This result was confirmed by flow cytometric analysis on PBMC from the patient. As shown on FIG. 3B the variant pattern of CD45 splicing can be identified by the absence of the single CD45R0+ T cell population. Taken together these results show that the patient exhibits abnormal CD45 splicing caused by the C77G polymorphism in the gene encoding CD45.

### Example 4

### Associations Between C77G and Hepatitis C

About 80% of those infected with HCV become chronic carriers, whereas the other 20% are able to clear the virus. In this example we analysed a cohort of HCV-infected individuals and showed that the presence of the exon 4 77G allele influences the ability to clear virus, with a relative risk of 7.2 for individuals carrying the 77G allele to become chronically infected.

Patients

DNA or serum samples from 388 patients were obtained from the Trent Hepatitis C Cohort study (Mohsen, A. H. and Group, T. H. (2001) Gut, 48, 707-13). The first 315 samples were selected randomly from a stored archive. Latterly, sera from 73 HCV RNA negative patients were specifically targeted for inclusion in this study. Of the total cohort, 256 patients were HCV RNA positive (Roche Amplicor assay), and the remaining 132 were HCV RNA negative in at least 2 serum samples. Amongst the 388 patients, 255 were males and 133 females. Most of the patients were Caucasoid except for 19 who were of other ethnic origins. All of the 15 C77G variant carriers identified in the present study were Caucasoid. 417 control genomic DNAs samples were taken from healthy British individuals (obtained from local blood donor banks or healthy donor volunteers) consisted of 236 and 181 samples previously described. Although these were from the UK, they were not demographically identical to the Trent cohort.

Functional and phenotypic analysis was performed on cryopreserved PBMC from healthy C77G and control C77C individuals obtained from local blood donor banks. The ages of all subjects in this study were between 20 and 58 years.

C77G Genotyping

To detect carriers of the 77G mutation, we used the amplification refractory mutation system (ARMS) PCR, with two separate reaction mixes, containing a common forward primer which is capable of amplifying both the wild type and the variant allele (in combination with a reverse primer) and one of the two reverse primers as follows:

| | |
|---|---|
| Ex4 fw | |
| 5′-CATATTTATTTTGTCCTTCTCCCA-3′ | (SEQ ID NO:6) |
| Ex4 revA | |
| 5′-GAAAGTTTCCACCAACGG-3′ | (SEQ ID NO:7) |
| Ex4 revB | |
| 5′-GAAAGTTTCCACGAACGC-3′ | (SEQ ID NO:8) |

The combination of Ex4 fw and Ex4 revA specifically amplifies the wild type allele only, whereas the combination of Ex4 fw and Ex4 revB specifically amplifies the variant allele only.

In some cases genotyping for C77G was carried out by Msp I digestion on PCR amplified exon 4 as described in Example 1. The presence of the C77G variant allele in all of the heterozygous samples was confirmed by sequencing.

Flow Cytometric Analysis

Surface phenotypic analysis was performed on cryopreserved PBMC from healthy C77G carriers and C77C controls. 2×10⁵ PBMC were stained with either allophycocyanine (APC)-conjugated CD4 (S3.3, Caltag, Silverstone, UK) or CD8-APC (clone RPA/T8, Pharmingen, San Diego, Calif.) along with fluorescein isothyocyanate (FITC)-conjugated CD45RA (clone HI100, Pharmingen) and phycoerythrin (PE)-conjugated CD45R0 (clone UCHL1, Pharmingen) mAbs in a single step at 4° C. for 20 minutes and washed with PBS, containing 0.2% BSA. The following reagents and antibodies were also used to stain cell suspensions: CD11a-FITC (G43-25B), CD28-FITC (CD28.2), CD62L-FITC (Dreg56), (DX2), CCR7 (2H4) all from BD Biosciences, Oxford, UK). Murine lymphocytes were stained with CD4-FITC (GK1.5), CD8-PerCP(53-6.7), CD44-PE (IM7), CD122-PE (Tm-α1) all from BD Biosciences and CD62-L (Mel-14) from Caltag, Silvertone, UK.

Statistical Analysis

The Fisher exact test was used to analyse the association of C77G polymorphisms and hepatitis C.

Results

We analyzed the frequency of the C77G allele in a cohort of 388 UK patients with Hepatitis C (HCV) infection and found 15 C77G heterozygotes (allele frequency, 1.9%), twice as high as in a UK control population of 417 individuals (0.95%). We next analyzed whether there is an association of the C77G variant with the outcome of infection. We compared the frequency of C77G in patients with chronic HCV infection (ie HCV RNA positive) with individuals who spontaneously cleared infection (anti-HCV positive, HCV RNA negative). We found 14 C77G heterozygotes among 256 chronic carriers (allele frequency, 2.7%) compared to 1 out of 132 in the anti-HCV positive, HCV RNA negative group (allele frequency 0.3%). The difference between the patients with chronic HCV infection and those with spontaneous resolution of infection is significant (p=0.02) and corresponds to a relative risk of 7.2 (95% CI=0.95-54.3) for inability of the C77G individuals to clear the virus. Taken together these results show a significant effect of the C77G allele in HCV progression to chronicity or clearance of virus.

TABLE 1
Frequency of CD45 exon 4 C77G variant in diseased and control groups.
The allele fequency is indicated in brackets. Statistically significant
differences between the diseased and control groups are indicated by
asterisks. In the case of hepatitis C the patients were HCV carriers
who cannot clear the virus and the control group were HCV patients who
resolved the infection and became HCV RNA negative.

| Population | Patient total No. | Control total No. |
|---|---|---|
| (reference) | (allele frequency %) | (allele frequency %) |
| Autoimmune hepatitis | | |
| Germany | 178 (3.2)* | 207 (0.7) |
| Multiple sclerosis | | |
| Germany | 327 (3.2)* | 303 (0) |
| Germany | 76 (3.3)* | 119 (0.4) |
| Italy | 194 (1.3)* | 222 (0) |
| Langerhans cell histiocytosis | | |
| Italy | 41 (3.7)* | 199 (0.3) |
| Hepatitis C | | |
| UK | 243 (2.9)* | 126 (0.4) |
| | (HCV carriers) | (HCV resolvers) |
| HIV | | |
| UK | 197 (2.9)* | 236 (0.85) |
| Systemic sclerosis | | |
| Germany | 67 (3.7)* | 207 (0.7) |

### Example 5

### Phenotypic Analysis of C77G Polymorphism

The following study examined whether the abnormal pattern of CD45 isoform expression affects other aspects of leukocyte phenotype.

Materials and Methods

Flow Cytometric Analysis

PBMC were stimulated for 12 days with 1 μg/ml of PHA-P (Sigma, St Louis, Mo.) adding IL-2 on day 10. Flow cytometric analysis of CD45 variant splicing was performed as previously described (Tchilian E Z, et al., ibid.). Briefly, 2×10⁵ PBMC were stained with either APC-conjugated CD4 (S3.3, Caltag, Silverstone, UK) or CD8-APC (clone RPA/T8, Pharmingen, San Diego, Calif.) along with FITC-conjugated CD45RA (clone HI10, Pharmingen) and PE-conjugated CD45R0 (clone UCHL1, Pharmingen) mAbs in a single step at 4° C. for 20 minutes and washed with PBS, containing 0.2% BSA. The following reagents and antibodies were also used to stain cell suspensions: CD11a-FITC (G43-25B), CD28-FITC (CD28.2), CD44-FITC (G44-26), CD62L-FITC (Dreg56), CD95-FITC (DX2), CCR7 (2H4) were all from PharMingen, CD62L-FITC (LAM 1-116) (Ancell, Bayport, USA), CD69-FITC (CH14) (Caltag, Silverstone, UK), HLA-DR-FITC (TU36) (Caltag), CD25-FITC (ACT-1) (Dako), CD4-FITC (Dako), CD27-FITC (LT27) (Serotec, Kidlington, UK). Isotype matched niAbs were used as controls. 10,000 or 50,000 events per sample were collected on a FACSCalibur (Becton Dickinson, Mountain View, Calif.) and analysed using WinMDI software.

Results

Cryopreserved PBMC's from healthy individuals known to carry the 77G mutation and cryopreserved normal control cells were analysed by flow cytometry. No apparent differences in the proportion of CD3, CD4, CD8, CD14 and CD19 cells were observed between the individuals with the 77G variant and wild type CD45 (data not shown). All of the 77G samples showed the previously described typical pattern of CD45 isoform expression on both CD4 and CD8 cells. Even after 12 days stimulation with PHA and IL-2, neither CD4 nor CD8 cells of 77G individuals were able to switch to expression of only the CD45R0 isoform (data not shown). However, it is noteworthy that the CD8 cells of individuals with the 77G mutation have more CD45RA single positive cells (mean 75%) compared to normal individuals (mean 58%) (p=0.001 for 6 77G carriers and 6 controls). In contrast the proportions of CD45RA versus CD45R0 or CD45RA/R0 double positive cells are similar among CD4 cells from 77G and control samples.

Because of the strikingly altered proportions of CD45RA positive versus CD45R0 or CD45RA/R0 double positive cells among CD8 cells from 77G and control individuals we next examined the expression of various cell surface markers associated with lymphocyte activation, analysing them in the CD45R0+ and CD45R0− subsets.

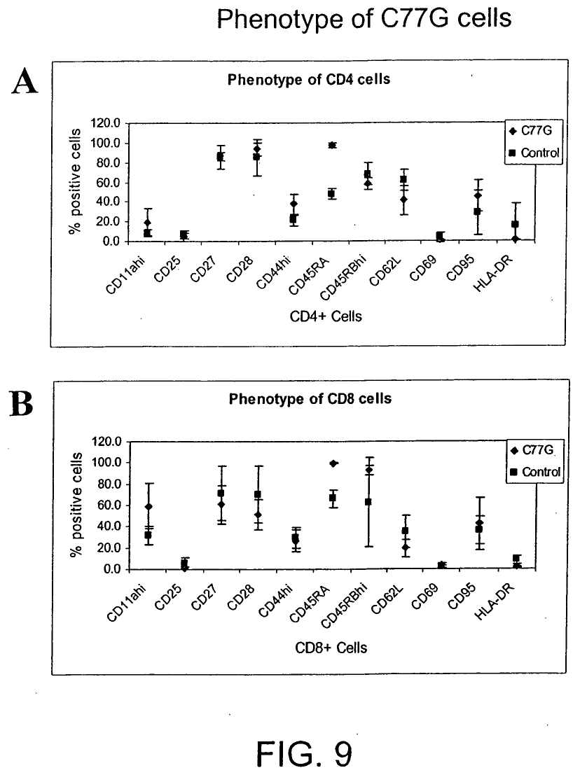

FIG. 9 illustrates FACs analysis of PBMC from 4 77G and 4 control normal individuals. Staining for a panel of markers has been analysed after gating on CD4 and CD8 T lymphocytes. In CD8 T cells no statistically significant differences were observed in the expression of the adhesion molecule CD44, the costimulatory molecule CD28, cytokine and chemokine receptors CD25 and CCR7 and the activation markers CD69 and HLA-DR. However, an increased frequency of CD8 cells expressing high levels of the adhesion molecule CD11a (CD11ahi) was detected in the (enlarged) CD45R0− subset (p=0.025). In the CD45R0+ subset the expression of CD27, CD62L and CD95 was significantly decreased in C77G individuals compared to controls. The differences in CD62L expression were confirmed with a different CD62L antibody (clone LAM1-116, data not shown), suggesting that the observed variances were not due to differential glycosylation of surface molecules in C77G and control cells.

### SUMMARY

Both CD4 and CD8 T cells show a decreased percentage of CD62L stained cells and an increased percentage of CD11ahi and CD95 positive cells in 77G individuals as compared to controls. These changes indicate that there are increased numbers of activated lymphocytes amongst both populations but the effects are more obvious in the CD8 population.

### Example 6

### Identification of A138G Polymorphism

Materials and Methods

Materials

314 Japanese genomic DNAs were collected from Osaka City University Medical School, Japan (69 of which were from patients with malignant gynaecological cancer). Peripheral blood mononuclear cells (PBMC) were isolated by centrifugation on a Ficoll-Paque (Amersham Pharmacia Biotech, Buckinghamshire, UK) density gradient and genomic DNA was extracted using DNA blood Minikit (Qiagen K. K., Tokyo, Japan). 209 Ugandan samples were provided by J. Whitworth and A. Hill (Wellcome Trust Centre for Human Genetics, Oxford, UK) (Tchilian, E. Z. et al., Immunogenetics 53: 980-983 (2002). 181 genomic DNA from British individuals consisted of 96 samples obtained through the local Blood Bank of the UK National Blood Transfusion Service, London, UK and 85 provided by Cancer & Immunogenetics Laboratory (Cancer Research UK, Oxford, UK). 72 Orkney samples were provided by Cancer & Immunogenetics Laboratory (Cancer Research UK, Oxford, UK), 48 Korean samples by J. C. Kim (College of Medicine and Asan Medical Centre, University of Uslan, Seoul), 74 Russian and 65 Tatar samples by Russian Rusibakiev (Academy of Science, Tashkent, Uzbekistan). Ethical approval was obtained and the patients gave consent for the study.

Denaturing High Performance Liquid Chromatography (DHPLC) and Sequencing

Genomic DNA was amplified by PCR using the following primers flanking the relevant exons: ex4 forward (5′-CATATTTATTTTGTCCTTCTCCCA-3′ (SEQ ID NO:6)) and ex4 reverse (5′-GTGCAGAAATGCAGGAAAT-3′ (SEQ ID NO:9)), ex6 forward (5′-GGAGAAGTGCTTGAAGATT-3′ (SEQ ID NO:1)) and ex6 reverse (5′-GTGCCAGATATTATTTGTAGG-3′ (SEQ ID NO:10)), generating fragments of 384 and 372 bps respectively. A two stage 34 cycle PCR was performed which included an initial 10 min denaturation at 95° C., then 14 cycles of 30 s at 95° C., 30 s at 61.5° C., 30 s at 72° C., followed by 20 cycles of 30 s at 95° C., 30 s annealing at 54° C. for exon 4 and 58° C. for exon 6, 30 s at 72° C., and a final 6 min extension at 72° C. PCR reactions were performed in a volume of 50 μl, containing 10 pmol of each primer, 200 μM dNTP, 2.5 mM MgCl₂ and 0.5 U of Amplitaq Gold (Perkin Elmer Life Sciences, Boston, Mass.) in 1× KCL Perkin Elmer buffer II. PCR products were resolved on 2% agarose, then hybridised for 4 min at 95° C., followed by 42 cycles of 1 min at 95° C. dropping by 1.6° C./cycle. Products were run on the DHPLC machine (Transgenomic WAVE, Transgenomic Ltd, Crewe, UK). Purified PCR products were subjected to automated sequencing using the same primers as for DHPLC.

Amplification Refractory Mutation System (ARMS) PCR

To detect carriers of the exon 4 C77G and exon 6 A138G mutations, we used amplification refractory mutation system (ARMS) PCR, with two separate reaction mixes, containing one forward primer and one of the two reverse primers. For exon 4 the original forward primer was used (5′-CATATTTATTTTGTCCTTCTCCCA-3′ (SEQ ID NO:6)) amplifying both the wild type and the variant allele. The reverse primer ex4 rev A (5′-GAAAGTTTCCACCAACGG-3′ (SEQ ID NO:7)) amplified only the wild type allele, while ex4 rev B (5′-GAAAGTTTCCACGAACGC-3′ (SEQ ID NO:8)) amplified only the variant allele. Similarly for exon 6 the original forward primer was used (5′-GGAGAAGTGCTTGAAGATT-3′ (SEQ ID NO:1)) and ex6 rev A (5′-CGTATCAGTCTGGACTCCA-3′ (SEQ ID NO:2)) to amplify the wild type or ex6 B (5′-CGTATCAGTCTGGACTCCG-3′ (SEQ ID NO:3)), amplifying the mutant allele only. Annealing temperatures were 56° C. for C77G and 60.5° C. for A138G. ARMS PCR products were resolved by Alkaline-Mediated Differential integration (AMDI) (Bartlett, S., Straub, J., Tonks, S., Wells, R. S., Bodmer, J. G. & Bodmer, W. F. (2001) Proc Natl Acad Sci USA, 98, 2694-2697). Samples were quantitated on a BMG Fluorostar plate reader. A random subset of samples was checked on 2% agarose gel.

RT-PCR

Total RNA was extracted from PBMC before and after the stimulation with PHA, using Tri-Reagent (Sigma, Dorset, UK). First-strand cDNA synthesis was performed using random hexadeoxynucleotide primers and the first strand cDNA synthesis Kit (Amersham Biosciences, Amersham, UK). The CD45 cDNA was amplified using primers spanning the alternatively spliced CD45 exons - ex2 forward primer (5′-CGAAGCTTGCTGTTTCTTAGGGACACG-3′ (SEQ ID NO:11)) and ex7 reverse (5′-GTGAATTCCAGAAGGGCTCAGAGTGGT-3′ (SEQ ID NO:12)). The PCR conditions for amplification of CD45 cDNA included 4 min incubation at 94° C. followed by 30 reaction cycles (1 min at 94° C., 1 min at 55 C, 4 min at 72° C.) and final 16-min extension at 72° C. The PCR products were resolved on a Visigel Separation Matrix (Stratagene, La Jolla, Calif.). Bands were quantitated using Quality One Software (Bio-Rad, Hertfordshire, UK).

Flow Cytometric Analysis

PBMC were surface stained with the following mAbs against human CD45 isoforms—CD45R0-PE (clone UCHL1, Pharmingen, San Diego, Calif.), CD45R0-FITC (clone UCHL1, Pharmingen) CD45RB-FITC (clone PD7/26, Dako, Glostrup, Denmark), CD45RB-PE (clone MT4, Pharmingen), CD45RA-FITC (clone HI10, Pharmingen), CD45RA-PE (clone 4KB5, Dako) along with APC-conjugated CD3 (Pharmingen). For CD45RC (clone YTH80.103, BioSource, Camarillo, Calif.) analysis a second layer of affinity purified F(ab)′₂ goat anti-rat FITC or PE (Caltag, Silverstone, UK) was used. Isotype matched mAbs were used as controls. 10,000 events per sample were collected on a FACSCalibur flow cytometer (Becton Dickinson, Mountain View, Calif.) and analysed using WinMDI software. For stimulation studies PBMC were stimulated for 12 days with 1 μg/ml of PHA-P (Sigma).

Results

Identification of a Novel Point Mutation in Exon 6 of CD45 in Japanese and Korean Populations

To examine the CD45 locus for novel polymorphisms, we used DHPLC to detect mutations in the alternatively spliced exons 4(A), 5(B) and 6(C) of CD45, followed by sequencing of the target individuals.

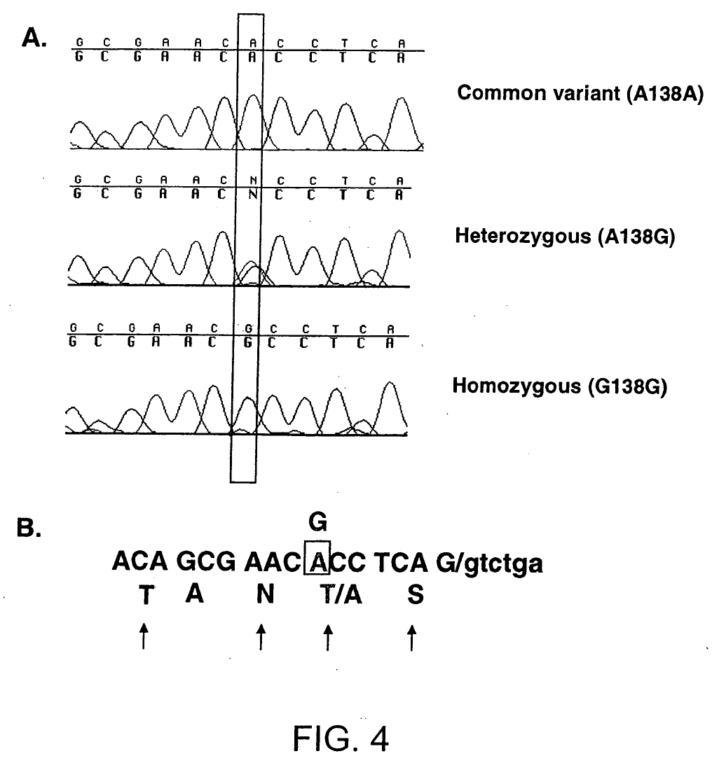

An A to G transversion at position 138 in exon 6 was found in Japanese samples. This is located 7 bp before the splice donor site at the 3′ end of exon 6, and results in a Threonine to Alanine semi-conservative amino acid change at position 47 of the CD45RC exon 6 (FIG. 4). Thr 47 is a potential O-linked glycosylation site (Van den Steen, P., Rudd, P. M., Dwek, R. A. & Opdenakker, G. (1998) Crit Rev Biochem Mol Biol 33, 151-208), but is also adjacent to an Asparagine and forms part of a consensus flanking sequence for an N-linked site as well. A substitution of this Thr may therefore lead to changes in the glycosylation of the extracellular domain of the molecule.

We used ARMS-PCR to detect the presence of the A138G variant and found that 130 individuals out of 314 Japanese samples carried the variant allele of which 13 were homozygotes for the G allele (allele frequency of 22.8%). The number of homozygotes was as expected according to the Hardy Weinberg Law. Note that the frequency of the A138G variant amongst the 69 Japanese patients with gynaecological cancer was within the normal range (27 heterozygotes and 4 homozygotes) and the presence of the variant did not correlate with any distinctive clinical manifestation. The high frequency of this allele in the Japanese population was further confirmed by re-sequencing all individuals indicated as carrying the allele. We also found 7 heterozygotes out of 48 Korean samples (allele frequency of 7.3%). The A138G variant was not detected in 209 Ugandan samples. We found 1 heterozygote out of 181 UK samples and 1 out of 72 Orkney samples. We also analysed samples from Asia and found 6 A138G heterozygotes in 65 Tatars (from Kazan and the Crimea) but none in 74 Russians from Tashkent. The 138G allele is also been found to be present in Chinese, Thai, Indian, Cambodian and Vietnamese individuals. 159 samples from Kagoshima in Southern Japan were also tested and revealed 114 A138A homozygotes, 36 A138G heterozygotes and 9 G138G homozygotes, a similar frequency of the 138G allele to that found previously in samples from Osaka.

We further compared the distribution of exon 6 A138G and exon 4 C77G variants, the latter being the only described common polymorphism in CD45 causing abnormal CD45 splicing. The C77G variant was absent in samples from an African population (Ugandan) as has been previously shown (Tchilian, E. Z., et al., (2002) Immunogenetics 53, 980-983.) and was not detected amongst the Far Eastern Japanese and Korean populations. Interestingly the exon 4 C77G variant was found at a higher frequency (3.5%) in the United Kingdom Orkney islands than elsewhere, but no C77G homozygotes were found in the samples studied.

In order to investigate the frequency of the 138G allele in other populations genotyping was carried out in 270 Thai, 20 Cambodian and 19 Peruvian Quechua Indians. The 138G allele was found to occur in the Thai and Cambodian populations with allele frequencies of 18.4% and 20%, respectively. The 138G allele was also be found to occur in a large cohort of Chinese individuals with a frequency of 21%. These data indicate that all Far Eastern Oriental populations tested have a similar distribution of the 138G allele. The 138G allele is not found in cohorts of Africans or West Europeans, but interestingly it was found to occur in samples from Peruvian Quechua Indians with a similar frequency of 21%.

CD45 Isoform Expression on PBMC of Individuals with the Exon 6 A138G Variant

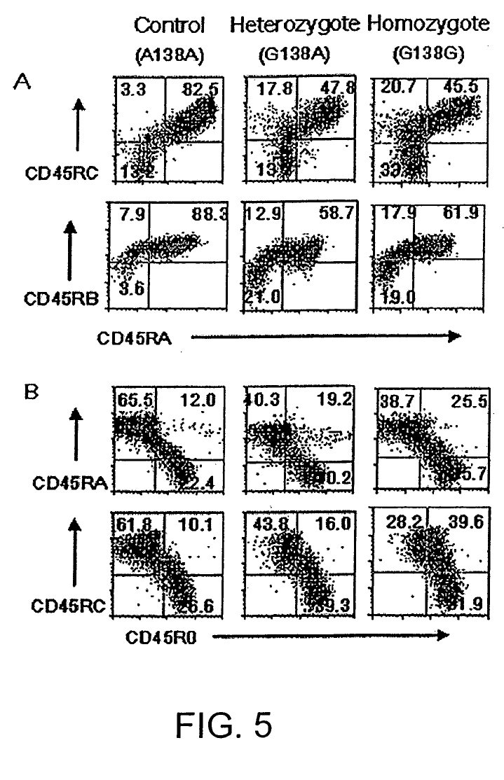

We next examined whether the novel A138G polymorphism affects CD45 isoform expression on the cell surface. Cryopreserved PBMC's from 4 healthy A138G heterozygotes, 4 G138G homozygotes and 4 common variant A138A homozygous controls were analysed by flow cytometry. CD45RA, CD45RB, CD45RC and CD45R0 antibodies were used to determine the expression of CD45 isoforms on these samples. There was a marked decrease in the proportion of cells expressing CD45RA and CD45RC or CD45RA and CD45RB isoforms in A138G positive individuals, with homozygotes showing a more extreme change (mean of 41.5% and 56.3% for CD45RA+CD45RC+ and CD45RA+CD45RB+ respectively) from the common variant controls 73.7% and 71.3% for CD45RA+CD45RC+ and CD45RA+CD45RB+) than the heterozygotes (49.6% and 55.9% for CD45RA+CD45RC+ and CD45RA+CD45RB+) (Table 2). There was a corresponding increase in CD45RA−CD45RC− or CD45RA−CD45RB− cells. Representative profiles are shown in FIG. 5A. A138G homozygotes had a higher proportion of cells expressing CD45R0 either in association with CD45RA (49.8% versus 22.8% in controls) and CD45RC (40.6% versus 11.2% in controls or in the absence of CD45RA (31.4% CD45R0+CD45RA− versus 20.5% in controls) and CD45RC (36.1% CD45R0+CD45RC− versus 20.1% in controls) (Table x, FIG. 5B).

After 11 days stimulation with PHA, all of the CD3+ cells of the A138G homo- or heterozygous individuals showed very similar phenotypes to common variant control individuals with predominant expression of CD45R0 and CD45RB isoforms (data not shown).

No differences were observed in isoform expression on CD3 negative cells (not shown).

Taken together these data suggest that exon 6 A138G carriers have fewer T cells expressing isoforms containing the A, B or C exons (naive phenotype cells) and have more activated CD45R0+ cells compared to the common variant CD45 controls.

Effect on CD45 Splicing.

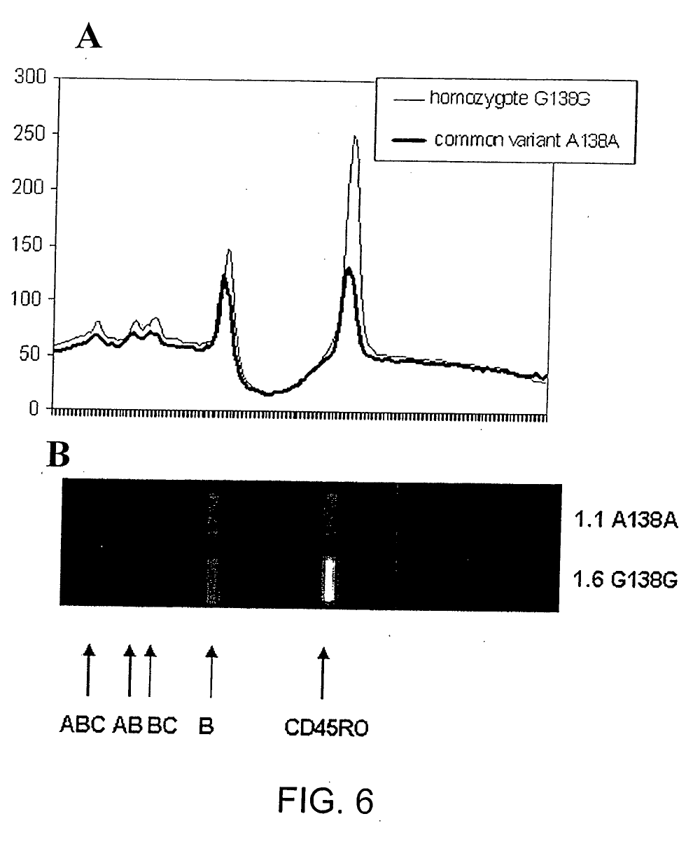

Because of the dramatic changes in the proportion of T cells expressing CD45 isoforms in A138G carriers, we next wanted to establish whether the exon 6 A138G variant interferes with CD45 splicing. RT-PCR analysis was performed on PBMC before and after stimulation with PHA. No qualitative differences in the expression of CD45 isoforms were observed between the homo-, and heterozygous A138G individuals and the controls at either time point (FIG. 6). However quantitation of the intensity of the bands showed a significant difference, in that the level of CD45R0 was increased in the mutated A138G gene when compared to the common variant.

These results suggest that the effect of this polymorphism is quantitative rather than qualitative with A138G carriers expressing more CD45R0 transcript compared to the controls.

Discussion

Described here is the identification of a polymorphism in exon 6 A138G of the gene encoding CD45 (PTPRC) which results in a semi-conservative amino acid substitution Thr47Ala in the extracellular domain of the CD45 molecule. This variant allele is present with a relatively high frequency in far Eastern populations, including Korean (7.3%), Japanese (22.8%), Chinese (21%), Thai (18.4%), Cambodian (20%) and Vietnamese (15.2%) populations. The G allele has also been shown to be present in India (8.3%) and Peruvian Indians (21%).

Phenotypic and functional analysis on A138G individuals indicates that the carriers of the A138G mutation have a higher proportion of CD45R0+ T cells and a decrease in naïve phenotype T cells expressing A, B and C isoforms.

The altered CD45 isoform expression is most likely caused by changes in alternative splicing, as shown by the increased levels of CD45R0 transcripts detected by RT-PCR in the A138G carriers. These findings are in agreement with earlier studies (Tsai, A. Y., Streuli, M. & Saito, H. (1989) Mol Cell Biol 9, 4550-4555) showing that mutations of nucleotides 134 to 144 at the most 3′ end of exon 6 resulted in mRNA that did not include exon 6 sequences. The exon 6 A138G mutation described here, exerts a more subtle quantitative effect and does not induce complete splicing out of exon C. It is plausible that in a similar way to the model proposed by Tsai et al., the A138G substitution may reduce the overall similarity of the splice site to the consensus sequence resulting in a less efficient recognition by the spliceosome. Alternatively, the exon 6 A138G change may induce alterations in exon splicing by disrupting regulatory elements within the exon itself (Smith, C. W. & Valcarcel, J. (2000) Trends Biochem Sci 25, 381-388). For example, the C77G polymorphism in exon 4 functions by disrupting an exon splicing silencer which normally represses the use of the 5′ splice site of exon 4 (Lynch, K. W. & Weiss, A. (2001) J Biol Chem 276, 24341-24347).

An alternative explanation for the observed phenotypic differences of the PBMC of A138G carriers might be that the variant results in the expression of a structurally altered CD45 molecule. Thus the A138G polymorphism results in the substitution of Thr47Ala, a potential glycosylation site for both O- and N-linked sugars and may therefore change the reactivity with carbohydrate dependent epitopes of anti-CD45 monoclonal antibodies (Pulido, R., Schlossman, S. F., Saito, H. & Streuli, M. (1994) J Exp Med 179, 1035-1040). Changes in the glycosylation would not only change the interactions with anti-CD45 antibodies, but may have important implications for the function of CD45 since the crucial contribution of carbohydrates to the regulation of CD45 isoform function has been documented. Several lectin like molecules have been shown to bind to carbohydrates of CD45, among other ligands. These include CD22 (Stamenkovic, I., Sgroi, D., Aruffo, A., Sy, M. S. & Anderson, T. (1991) Cell 66, 1133-1144), galectins (Perillo, N. L., Pace, K. E., Seilhamer, J. J. & Baum, L. G. (1995) Nature 378, 736-739; Walzel, H., Schulz, U., Neels, P. & Brock, J. (1999) Immunol Lett 67, 193-202; Symons, A., Cooper, D. N. & Barclay, A. N. (2000) Glycobiology 10, 559-563), mannose receptor (Martinez-Pomares, L., Crocker, P. R., Da Silva, R., Holmes, N., Colominas, C., Rudd, P., Dwek, R. & Gordon, S. (1999) J Biol Chem 274, 35211-35218) and serum mannan binding protein (Uemura, K., Yokota, Y., Kozutsumi, Y. & Kawasaki, T. (1996) J Biol Chem 271, 4581-4584; Baldwin, T. A. & Ostergaard, H. L. (2001) J Immunol 167, 3829-383). The CD45 ectodomain has also been suggested to influence CD45 engagement in cis interactions with TCR, CD4 and CD5 (Alexander, D. R. (1997) In Lymphocyte Signalling: Mechanism, subversion and manipulation, eds Harnett, M M & Rigley, K P John Wiley & Sons Ltd., 107; Leitenberg, D., Y. Boutin, D. D. Lu, and K. Bottomly (1999) Immunity 10, 701; Dornan, S., et al., J Biol Chem 277, 1912-1918), but no direct binding between the CD45 ectodomain and another protein has been shown so far. Another proposed role for the CD45 extracellular domain is the regulation of dimersation and there is evidence that CD45 forms dimers on the cell surface (Majeti, R., et al.,(2000) Cell 103, 1059-1070; Xu, Z. & Weiss, A. (2002) Nat Immunol 3, 764-771). These studies suggest that the structural differences caused by the A138G variant could affect the interactions of CD45 with potential ligands in cis and trans as well as dimerisation between CD45 isoform and might have functional consequences for the immune response.

It is interesting that the exon 4 C77G and exon 6 A138G variants have different distributions. This may suggest that variants arose independently after the emigration of ancestral humans from Africa. The high frequency of A138G variant in Japan suggests that it arose in the Far East and its low frequency elsewhere would confirm this. The maintenance of these CD45 variants in different human populations may be ascribed at present to selection or drift. Further functional and disease association studies may provide more convincing evidence for a selective effect, particularly of the novel A138G variant.

In Caucasoids the commonest polymorphism with an obvious phenotypic effect is the previously described C77G mutation in exon 4, which prevents normal splicing from high (CD45RA) to low (CD45R0) molecular weight isoform. We have previously shown that the frequency of the C77G variant allele in Northern Europe and North America is in the region of 0.85 to 1.6% and that it is absent in Africans (Tchilian, E. Z., et al., (2002) Immunogenetics 53, 980-983). The data presented here confirm the previously observed frequency in the UK (on a different set of UK samples) and the lack of this variant in African Ugandan populations (Tchilian, E. Z., et al., (2002) Immunogenetics 53, 980-983), and indicate a similar lack amongst the Far Eastern Japanese and Korean populations. The only exceptions are individuals from the UK Orkney Islands who have a higher allele frequency for the variant (3.5%). There may be an association between the increased prevalence of exon 4 C77G variant and the high incidence of multiple sclerosis in the Orkney islands.

In summary the results suggest that individuals with the A138G variant have an increased proportion of T cells with an activated, memory or effector phenotype as determined by the increased proportion of CD45R0+ cells and reduced number of cells expressing the CD45 A, B and C isoform. The altered CD45 expression may therefore contribute to changes in interaction with potential ligands or homo- or heterodimerisation of the CD45 isoforms. Xu & Weiss (2002) Nat Immunol 3, 764-771 recently proposed a model suggesting that expression of the CD45R0 isoform in activated cells shifts the equilibrium of cell surface CD45 towards dimers, and acts as a negative regulator, contributing to the cessation of the immune response. Increased expression of the CD45R0 isoform caused by A138G polymorphism would promote this negative regulation, resulting in a less vigorous immune response which may reduce the risk of autoimmune disease in A138G carriers. Alternatively, the high proportion of CD45R0+ cells may indicate that these individuals have a larger memory population and can make vigorous recall responses to pathogens. The high frequency of this allele in Japan and Korea may indicate that it confers a survival advantage.

TABLE 2
CD45 isoform expression on CD3+ cells from A138G and control
individuals. Means and standard deviations of data expressed as the
percentage of CD3+ T cells from 4 homozygous (G138G),
4 heterozygous (A138G) and 4 homozygous for the common variant
(A138A) control individuals.

| | Control | Heterozygote | Homozygote |
|---|---|---|---|
| Cell Subset | (A138A) | (G138A) | (G138G) |
| CD45RA+CD45RC+ | 73.7 +/− 12.0 | 49.6 +/− | 41.5 +/− 5.5 |
| CD45RA−CD45RC− | 18.7 +/− 8.4 | 34.5 +/− | 36.0 +/− 3.7 |
| CD45RA+CD45RB+ | 71.3 +/− 14.1 | 55.9 +/− | 56.3 +/− 5.5 |
| CD45RA−CD45RB− | 10.1 +/− 7.8 | 20.7 +/− | 21.8 +/− 2.1 |
| CD45RC+CD45R0+ | 11.2 +/− 5.4 | 13.4 +/− | 40.6 +/− 5.7 |
| CD45RC+CD45R0+ | 20.1 +/− 6.6 | 32.0 +/− | 36.1 +/− 5.0 |
| CD45RA+CD45R0+ | 29.8 +/− 22.8 | 56.5 +/− 12.5 | 49.8 +/− 23.3 |
| CD45RA+CD45R0+ | 20.5 +/− 78 | 31.8 +/− 6.0 | 31.4 +/− 3.6 |

### Example 7

### Associations Between A138G and Disease

Methods

Materials

DNA samples from 175 Graves' disease (GD) and 126 Hashimoto's thyroiditis (HT) patients were obtained through Osaka City University Hospital. Hyperthyroidism due to Graves' disease was diagnosed on the basis of history and signs of hyperthyroidism with diffuse goiter and the laboratory findings, including elevated serum free T4 and free T3 concentrations, undetectable serum thyroid stimulating hormone (TSH), and positive TSH receptor antibody. Hashimoto's thyroiditis was diagnosed by positive thyroglobulin and/or thyroid peroxidase antibodies, reduced echogenicity on thyroid ultrasound, and normal or elevated TSH level.

128 Hepatitis B and 286 Hepatitis C samples were collected in the outpatient clinic of Osaka City University Hospital. All of the 128 Hepatitis B patients were infected at birth by transmission from their mothers and were positive for Hepatitis B surface antigen. The Hepatitis C patients were infected later in life and were all positive for antibodies to HCV antigen. In all samples HCV RNA was detected, except for four patients who have cleared the virus (two of these were A138G heterozygous and one G138G homozygous). As control samples 314 Japanese genomic DNA's collected from Osaka City University Medical School were used as previously described in Example 6. Approval was obtained by the Ethical committee of the City University Graduate School of Medicine Osaka and the patients gave consent for the study.

Genotyping

Two different genotyping techniques were used to detect carriers of the exon 6 A138G mutations.

(1) Amplification refractory mutation system (ARMS) PCR, with two separate reaction mixes, containing one forward primer and one of the two reverse primers was carried out as previously described (Example 6 and Stanton, T. et al. Proc Natl Acad Sci USA 100, 5997-6002. (2003)).

(2) AMDI which is based on alkaline-mediated detection of ARMS-PCR products using double-stranded DNA specific dyes (Bartlett et al. 2001, PNAS USA, 98: 2694-7; Stanton et al. 2003, PNAS USA, 100: 5997-6002). Two separate reaction mixes containing the forward (5′-GCAGAAGTGCTTGAAGATT (SEQ ID NO:19)) and one of the two reverse primers (5′-GCATAGTCAGACCTGAGCT (SEQ ID NO:20) for the A allele and 5′-GCATAGTCAGACCTGAGCC (SEQ ID NO:21) for the G allele) were used. Annealing temperatures were 52° C. and 55° C. for the A and G allele respectively.

The presence of the 138G variant allele in all of the samples analyzed was confirmed by sequencing.

Flow Cytometric Analysis

Phenotypic analysis was performed on PBMC from 6 healthy A138A controls and 4 healthy G138G homozygous carriers. Cells from 4 A138G heterozygotes were also analysed (data not shown). The ages of all subjects in this study were between 27 and 58 years. 2×10⁵ PBMC were stained with either allophycocyanine (APC)-conjugated CD4 (S3.3, Caltag, Silverstone, UK) or CD8-APC (clone RPA/T8, Pharmingen, San Diego, Calif.) along with fluorescein isothyocyanate (FITC)-conjugated CD45RA (clone HI10, Pharmingen) and phycoerythrin (PE)-conjugated CD45R0 (clone UCHL1, Pharmingen) mAbs in a single step at 4° C. for 20 minutes and washed with PBS, containing 0.2% BSA. The following reagents and antibodies were also used to stain cell suspensions: CD11a-FITC (G43-25B), CD28-FITC (CD28.2), CD62L-FITC (Dreg56), CD95-FITC (DX2), CCR7 (2H4) were all from Pharmingen, CD27-FITC (LT27) (Serotec, Kidlington, UK).

For intracytoplasmic staining 1×10⁵ PBMC per well were incubated in U-bottom 96-well plates in 200 μl of RPMI1640+10% FCS in the presence of 50 ng/ml phorbol myristate (PMA) and 0.5 μg/ml ionomycin. GolgiPlug (Pharmingen) was added after 2 hours and cells incubated for an additional 12 hours. The cells were surface labelled with CD4-APC or CD8-APC antibodies as described above and permeabilised with 40 μl Permafix (OrthoDiagnostic, UK) for 40 min in the dark. The cells were washed and stained with FITC conjugated IFN-gamma antibody (clone 25723.11, Pharmingen) for 30 min at room temperature.

Isotype matched mAbs were used as controls. 10,000 or 50,000 events per sample were collected on a FACSCalibur (Becton Dickinson, Mountain View, Calif.) and analysed using WinMDI software.

Statistical Analysis

Chi-Square test, using Yates continuity correction to allow for small numbers was used to analyse the disease association of the 138G variant allele. For comparison of phenotypic analyses between cell subsets in A138G and control individuals, Student's t-test, assuming equal variance, was used.

Results

We studied the frequency of the 138G variant in cohorts of Japanese patients with thyroid autoimmune conditions. In Hashimoto's thyroiditis (HT) cellular and humoral responses to thyroid antigens lead to destruction of the organ and hypothyroidism while Graves' disease (GD) is characterised by hyperthyroidism, caused by stimulatory thyrotropin receptor antibodies. We analysed 126 Hashimoto patients and found 50 A138G heterozygotes (allele frequency 19.8%), comparable to the frequency in the control population (22.8%). Interestingly no G138G homozygotes were detected amongst the Hashimoto samples although 5 were expected according to the Hardy-Weinberg Law (p=0.02) a result suggesting a recessive effect of the 138G allele in HT. We found 31 heterozygotes (frequency 9%) and no homozygotes out of 175 Graves' samples. The difference between the controls and GD is very significant (p<0.01). In contrast to HT, this suggests a dominant effect for the 138G allele in GD.

We further analysed the frequency of the A138G variant in two important viral infections—Hepatitis B (HBV) and Hepatitis C. We found 20 A138G heterozygotes and no homozygotes among 128 Hepatitis B carrier samples (allele frequency, 12.6%). The difference between the controls and HBV is significant (p=0.0004). The reduction in heterozygotes suggests a dominant effect of the 138G allele in this disease.

In the case of hepatitis C, a reduced frequency of the 138G allele was found in HCV patients who cannot clear the virus, as opposed to “control” individuals who were infected with HCV but resolved the infection and became HCV RNA negative. The difference in the frequency of the 138G allele between patients and controls is statistically significant p=0.02.

A decreased frequency of the 138G allele was observed type I diabetes (16.8%, p=0.014).

Taken together these studies confirm that the 138G allele influences the incidence of infectious and autoimmune diseases. The 138G allele shows a significant dominant protective effect in GD and HBV infection, but a recessive protective effect in HT.

Immune Function and Phenotype

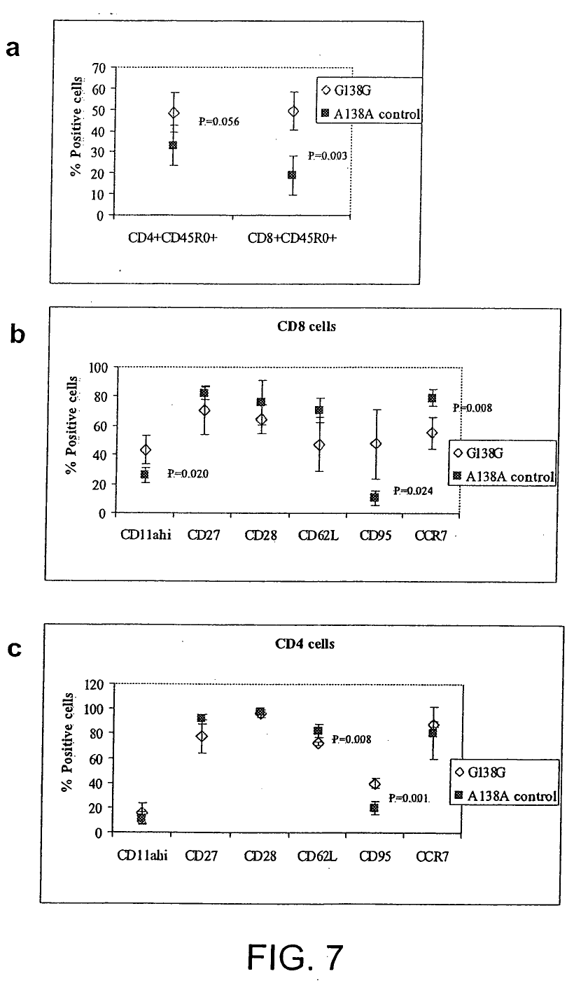

We next sought evidence for altered immune phenotype and function in individuals carrying the 138G allele. We examined whether the altered pattern of CD45 isoforms in 138G individuals affects the expression of other leucocyte antigens associated with differentiation of T cells. Peripheral blood mononuclear cells (PBMC's) from healthy G138G homozygotes, A138G heterozygotes and A138A control homozygotes were analysed by flow cytometry. All the G138G variant samples showed the previously described increased proportion of CD45R0+ T cells (Stanton, T. et al. Proc Natl Acad Sci USA 100, 5997-6002. (2003)); among CD8 cells mean 49.4±8.9%, compared to 18.9±9.3% in controls (p=0.003) (FIG. 7a), and in the CD4 subset mean 48.4±9.3 % versus 32.8±9.3% in A138A controls (p=0.056). A138G heterozygotes show an intermediate CD45R0+ phenotype for CD8 and CD4 cells (data not shown). Furthermore the G138G individuals exhibit decreased expression of CD27, CD28, CD62L and CCR7 and increased expression of CD11a and CD95 (FIG. 7b). Less exaggerated changes in expression of these markers were detected in the CD4 (FIG. 7c). These changes suggest that the most prominent effect in 138G individuals is an increase in the proportion of activated/memory T cells.

We next analysed cytokine production in PBMC from 138G individuals. Intracytoplasmic flow cytometric analysis showed that stimulated G138G cells have a significantly higher frequency of CD4 and CD8 cells able to secrete interferon-gamma (IFN-gamma) (Table 4). Heterozygotes showed an intermediate frequency of cytokine-producing cells in both T cell subsets. These results show that expression of the variant 138G allele is associated with increased production of the Th1 cytokine IFN-gamma by CD4 and CD8 cells.

TABLE 3
Frequency of CD45 exon 6 A138G alleles in control and disease groups
from Osaka (A) and other regions (B).

| Disease | Total | | | | % |
|---|---|---|---|---|---|
| Group | number | A138A | A138G | G138G | allele G frequency |
| (A) | | | | | |
| Japanese | 314 | 184 | 117 | 13 | 22.8 |
| controls | | | | | |
| Hepatitis B | 128 | 98 | 28 | 2 | 12.6*** |
| Hepatitis C | 286 | 194 | 82 | 10 | 17.8* |
| Diabetes | 190 | 132 | 52 | 6 | 16.8** |
| Cervical | 69 | 38 | 27 | 4 | 25.4 |
| cancer | | | | | |
| Hashimoto | 126 | 76 | 50 | 0 | 19.8 |
| Graves | 175 | 144 | 31 | 0 | 8.9+ |
| (B) | | | | | |
| Thailand | 270 | 179 | 76 | 11 | 18.4 |
| Cambodia | 20 | 12 | 8 | 0 | 20 |
| Peru | 19 | 12 | 6 | 1 | 21 |
| China | 560 | 349 | 187 | 24 | 21 |
| Vietnam | 451 | 322 | 121 | 8 | 15.2 |
| India | 222 | 188 | 31 | 3 | 8.3 |

Statistically significant differences from controls are indicated by the symbols as follows:
*p = 0.02,
**p = 0.017,
***p = 0.0004 and
+p < 0.0001.

TABLE 4
Frequency of IFN-gamma producing CD4 and CD8 T cells of individuals
with different 138G alleles.

| | % CD4 cells expressing | % CD8 cells expressing |
|---|---|---|
| | IFNγ | IFNγ |
| G138G | 30.7 +/− 4.1* | 35.3 +/− 11.6⁺ |
| A138G | 28.6 +/− 4.3** | 17.8 +/− 4.6⁺⁺ |
| A138A | 18.9 +/− 1.8 | 9.7 +/− 2.8 |

Four individuals of each genotype were studied. Differences between G138G or A138G individuals and A138A controls were analysed by Student's T test.
*p = 0.002,
**p = 0.006,
⁺p = 0.005,
⁺⁺p = 0.024.

Discussion

There are several possible explanations for the effect of the 138G variant. An important factor in the pathogenesis of autoimmune diseases is a change in the balance between Th1 cytokines which promote cell mediated immunity, and Th2 cytokines, which promote humoral immunity. In GD there is a shift toward Th2 cytokine responses (Kocjan, T., et al., Pflugers Arch 440, R94-95. (2000); Ludgate, M. & Baker, G. Clin Exp Immunol 127, 193-198. (2002)), while Hashimoto's patients show Th1 activation (Mazziotti, G. et al. Eur J Endocrinol 148, 383-388. (2003)). It is likely that the increased IFN-gamma production in 138G carriers would counteract the Th2 cytokine deviation in GD. Furthermore is has been suggested that activated (IFN-gamma-producing) CD8 cells may reduce the pathogenic Th2 dominance in GD (Bartlett, S. et al., (2001) Proc Natl Acad Sci USA 98, 2694-2697). In contrast the increased IFN-gamma production in the 138G variant might not affect the disease course and already polarised Th1 balance in HT. However, the lack of G138G homozygotes in HT suggests the possibility of a specific effect in homozygotes which needs further investigation.

The contribution of CD8 cells to the control of HBV infection is well documented (Thimme, R. et al. J Virol, Vol. 77, 68-76. (2003)). In addition to clearance of infected cells by cytolytic CD8 cells, the anti-viral effect of IFN-gamma produced by these cells has been shown to be an important protective mechanism (Guidotti, L. G. & Chisari, F. V. Annu Rev Immunol, 19, 65-91. (2001)). It is very likely that the increased proportions of activated T cells and IFN-gamma production in 138G neonates would limit amplification of the virus. Furthermore it has been suggested that neonates have Th2 biased immune responses (Chen, N. & Field, E. H. Transplantation, 59, 933-941. (1995); Barrios, C. et al. Eur J Immunol, 26, 1489-1496. (1996)) and is possible that the prevalence of Th1 cytokines in 138G infants would be beneficial at this early stage of life in controlling the HBV infection, while it would not have such a significant impact later in life. This might be the case for the Hepatitis C cohort we have studied. Whatever the mechanism, comparisons of immune responses of individuals carrying or lacking the 138G allele may provide insights into the molecular mechanisms underlying the interactions between HCV and HBV and IFN-gamma.

Although there have been previous reports of altered CD45 isoform expression in disease (Sempe, P. et al. Int Immunol, 5, 479-489 (1993); Renno, T. et al. Int Immunol, 6, 347-354 (1994); Subra, J. F. et al. J Immunol, 166, 2944-2952 (2001)), we now provide evidence that genetic variants affecting CD45 isoform expression are associated with autommunity and viral infection, suggesting a crucial role of CD45 in modulating immune responses. It is conceivable that the original selection for the 138G CD45 variant may have been with respect to pathogen resistance and what we see now is a residue of this after the pathogen effect has gone. The high frequency of 138G individuals (40% in Japan) suggests that the allele is likely to affect susceptibility and pathogenesis in other autoimmune and infectious diseases.

### Example 8

### Identification of Novel A54G Polymorphism

Materials and Methods

Materials:

269 Ugandan DNA samples from the Entebbe cohort (160 HIV-seropositive and 109 seronegative) were provided by Pontiano Kaleebu, Christine Watera, Jimmy Whitworth and Adrian Hill. 181 UK, 175 Japanese and 48 Korean genomic DNA samples were obtained as previously described (Stanton, T., et al., 2003, Proc Natl Acad Sci USA 100:5997.). 40 Malawian samples were provided by Paul Fine and Hazel Dockrell. All of these samples were HIV negative. Ethical approval was obtained. PBMC samples for FACS analysis were obtained from one A54G and two A54A control Ugandans only. All these were HIV positive.

Denaturing High-Performance Liquid Chromatography (DHPLC) and Sequencing.

Genomic DNA was amplified by PCR by using the following primers flanking exon 4: ex4 forward (5′-CATATTTATTTTGTCCTTCTCCCA-3′ (SEQ ID NO:6), ex4 reverse (5′-GTGCAGAAATGCAGGAAAT-3′ (SEQ ID NO:9)) generating a product of 384 bps. A two-stage, 34-cycle PCR was performed, with an initial 10-min denaturation at 95° C., then 14 cycles of 30 s at 95° C., 30 s at 61.5° C. minus 0.5° C. per cycle, and 30 s at 72° , followed by 20 cycles of 30 s at 95° C., 30 s of annealing at 54° C., 30 s at 72° C., and a final at 72° C. PCRs were performed in a volume of 50 μl, containing 10 pmol of each primer, 200 μM dNTP, 2.5 mM MgCl₂, and 0.5 units of AmpliTaq Gold (Perkin-Elmer) in 1× KCl Perkin-Elmer buffer II. 5 μl of the PCR product was resolved on 2% agarose to test product size, and the remaining product was denatured for 4 min at 95° C., followed by 42 cycles of 1 min at 95° C., dropping by 1.6° C. per cycle to 28.8° C. to hybridize. Products were run on the DHPLC machine (WAVE, Transgenomic, Crewe, U.K.). Purified PCR products were subjected to automated sequencing by using the same primers as for DHPLC.

Flow Cytometric Analysis

PBMC were surface stained with the following mAbs against human CD45 isoforms—CD45R0-PE (clone UCHL1, Pharmingen, San Diego, Calif.), and CD45RA-FITC (clone HI10, Pharmingen), along with APC-conjugated CD3 (Pharmingen). 10,000 events per sample were collected on a FACSCalibur flow cytometer (Becton Dickinson, Mountain View, Calif.) and analysed using WinMDI software.

Results

Using denaturing high performance liquid chromatography, an A to G transversion was found at position 54 in exon 4. This A54G variant results in a Thr to Ala semiconservative amino acid substitution at position 19 of the CD45RA exon 4.

The A54G variant was found in Ugandan samples, but was absent amongst Far Eastern (175 Japanese and 48 Koreans), UK Caucasoids (181 UK and 72 Orkneys) and African (40 Malawian) populations (Table 5).

We investigated the distribution of the new 54G allele in a Ugandan (Entebbe) cohort of HIV seropositive and seronegative individuals. We found six A54G heterozygotes out of 160 HIV seropositive (allele frequency, 1.87%) and one heterozygote out of 109 HIV seronegative controls individuals (0.45%). The presence of the variant allele was confirmed by sequencing in all samples. The difference between the controls and HIV infected individuals is very similar to that observed by us for the C77G variant in UK HIV infected individuals, with about four fold higher frequency in patients versus controls (p=0.24 by Fisher exact test).

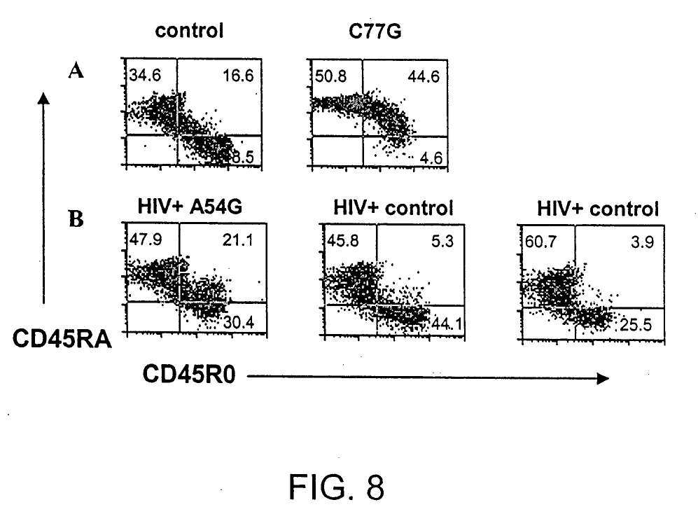

Since exon 4 C77G and C59A variants have been shown to alter CD45 splicing, we next examined whether the A54G polymorphism affects CD45 isoform expression on the cell surface. Cryopreserved PBMC from one A54G and 2 A54A control individuals were analysed, all three were HIV seropositive. PBMC samples were gated on CD3 T cells and CD45RA and CD45R0 expression analysed. The normal pattern of CD45 expression is characterised by the presence of CD45RA+ cells lacking CD45R0 expression and CD45R0+ cells lacking CD45RA (FIG. 8A), while the abnormal C77G pattern is characterised by the absence of the CD45RA− population and an increased proportion of double positive RA/RO cells (44.6 versus 16%). The A54G individual showed an increase proportion of double positive RA+R0+ cells, compared to the two Ugandan HIV A54A controls (21.1% versus 5.3% and 3.9%) (FIG. 8B), although there is no clear deficit of CD45RA− cells as in C77G.

TABLE 5
Frequency of A54G variant in Ugandan HIV seropositive and seronegative
individuals.

| Polymorphism | Population | Allele frequency (%) |
|---|---|---|
| Exon 4 A54G | HIV Ugandans | 1.87 |
| | Control Ugandans | 0.45 |
| | UK & Orkney | 0 |
| | Japanese | 0 |
| | Koreans | 0 |
| | Malawi | 0 |

### Example 9

### Construction of Transgenic Mice as Models of Expression Patterns CD45 C77G and A138G Human Variants

In order to investigate the role of distinct combinations of CD45 isoforms we constructed Tg mice expressing single CD45RABC or CD45R0 transgenes and a normally splicing allele (CD45RABC/+ and CD45R0/+) mimicking the expression of CD45 in C77G or A138G human variants.

Materials and Methods

CD45 Transgenic Mice

Transgenic mice expressing single CD45RABC or CD45R0 isoforms under the control of the human CD2 promoter have been described previously (Tchilian et al. Int Immunol. 16:1323-1332, 2004). The transgenic mice were bred onto CD45 homozygous knockout mice. Two lines with each isoform were constructed with high and low levels of transgene expression. These lines are referred to as CD45RABC^{hi}, CD45RABC^{lo}, CD45RO^{hi} and CD45RO^{lo}. CD45 Tg mice expressing two isoforms (CD45RABC×CD45RO) were generated by breeding CD45RABC^{hi} and CD45RO^{hi} mice.

We generated mice with one normally splicing CD45 allele and a fixed non splicing single CD45 transgene (CD45^{RABC/+} and CD45R0^{/+} mice) by breeding single isoform CD45RABC or CD45R0 Tg mice with C57B1/6 mice. The presence of the CD45 transgenes in the F1 crosses was detected by PCR on tail genomic DNA, using forward 5′-GAGCTCAGAATCAAAAGAGGA-3′ (SEQ ID NO:22) and reverse 5′-TAATTCACAGTAATGTTCCCAAACATGGC-3′ (SEQ ID NO:23) primers, generating a 1000 bp and 710 bp products for the CD45RABC and CD45R0 transgenes respectively. All mice were bred in the specific-pathogen free facilities of the Institute for Animal Health, Compton, UK and used for experiments at 6-8 weeks of age unless otherwise stated. All experiments fully complied with relevant Home Office guidelines and were approved by the animal ethical committee of the Institute for Animal Health.

Flow Cytometric Analysis

The following reagents and antibodies were also used to stain cell suspensions: CD4-FITC (GK1.5), CD8-PE (53-6.7), pan CD45-CyC (30-F11), CD45RA-biotin (14.8), all from BD Biosciences. Apoptosis was visualised by Annexin V staining. Cells were first surface stained using CD4 and CD8 antibodies, followed by Annexin staining according to the manufacturers instruction (Pharmingen, Oxford, UK).

T Cell Activation

For T cell stimulation, 2×10⁵ lymph node cells were placed into round bottom 96 well plates. Phorbol 12-myristate 13-acetate (PMA, 50 ng/ml) and calcium ionophore (ionomycin, 200 ng/ml) were added to the wells. For TCR-CD3 crosslinking, the plates were coated overnight at 4° C. with varying concentrations (from 0 to 10 μg/well) of anti-mouse CD3ε(clone 145-2C11, BD Biosciences) in the presence or absence of 1 μg/well of CD28 (clone 37.51, BD Biosciences) and washed three times before incubation with the T cells. For mixed lymphocyte reactions (MLR), 2×105 lymph node cells of CD45 transgenic H-2b mice were placed into round bottom 96 well plates in the presence of 2×10⁶ (10:1), 1×10⁶ (5:1) and 2×10⁵ (1:1) T cell-depleted Balb/c H-2d stimulator cells. These were prepared by treating the splenocytes with anti-Thy1.2 mAb, followed by addition of guinea pig complement and irradiating with 2,500 rad. T cells were harvested at various intervals (24-148 h) after a 12 h pulse with 1 μCi of [³H] thymidine per well.

Cytokine Production

Cytokine production was measured using BD Biosciences Mouse Th1/Th2 Cytokine Cytometric Bead Array (CBA) kit, following the manufacturer's protocol. Briefly for each test sample, 10 μl of each cytokine capture bead suspension was mixed and 50 μl of the mixed beads were transferred to each assay tube, followed by 50 μl of PE detection reagent and 50 μl of diluted standard or neat sample. The samples were incubated for 2 hours at room temperature, in the dark, washed and resuspended in 300 μl of wash buffer. The standards and test samples were analysed on a FACSCalibur (Becton Dickinson, UK), using CellQuest and BD CBA software, in accordance with the manufacturer's instructions.

Western Blot Analysis

For total protein extraction, cell lysis (2×10⁶ cells/ml) was performed in RIPA (PBS pH 7.4, 1% NP-40, 0.5% Sodium deoxycholate, 0.1% SDS, 10 mM NaF and 0.5 mM PMSF) buffer, containing protease and phosphatase inhibitors (Protease inhibitor cocktail and Phosphatase inhibitor cocktail 2, Sigma-Aldrich, Dorset, UK), on ice at 4° C. for 30 min. Insoluble materials were removed by centrifugation (15,000× g for 10 min at 4° C.) and the total protein concentration of each sample was quantified by BCA protein assay (Pierce Biotechnology, Rockford, Ill.). Cell lysate proteins were analysed by 10% SDS-PAGE (10 μg of total protein per lane), transferred onto a nitrocellulose membrane and immunoblotted using antibodies specific for STAT1, pY701STAT1 and Lck (Upsate Biotechnology, Inc., N.Y.) or pY505Lck (Signalling Technologies, Beverly, Mass.). Blots were developed using ECLTM donkey anti-rabbit HRP linked F(ab′)2 fragment and ECL™ Western Blotting Detection Reagents (Amersham Biosciences, UK Ltd.).

Induction of Experimental Autoimmune Encephalomyelitis (EAE)

EAE was induced by subcutaneous injection of Myelin Oligodendrocyte Glycoprotein peptide (pMOG35-55—MEVGWYRSPFSRVVHLYRNGK (SEQ ID NO:24)) consisting of 200 μg peptide antigen in a final volume of 100 μl Complete Freund Adjuvant (CFA, Difco Laboratories, Detroit, Mich., USA) containing Mycobacterium tuberculosis (4 mg/ml). On the same day and 2 days later, all mice received two i.p doses of pertussis toxin (200 ng/injection; Sigma). Mice were scored twice daily for symptoms of EAE as follows: 0, no signs; 1, flaccid tail; 2, partial hind limb paralysis and/or impaired righting reflex; 3 full hind paralysis; 4, hind limb plus fore limbs paralysis; and 5, moribund and dead. The two scores for each day were added together and divided by the number of surviving mice to give the mean EAE grade for each day.

Results

Proliferative and Cytokine Responses in CD45Tg/+ Mice.

Because in humans expressing C77G or A138G CD45 variant alleles it is the combinations of isoforms expressed on cells that are altered we generated mice expressing one normally splicing CD45 allele and a CD45RABC or CD45R0 transgene. We constructed CD45^{RABC/+} mice (F1 of CD45RABC^{hi}×CD45^{+/+}) which mimic the expression in C77G variant humans and CD45R0^{/+} mice (F1 of CD45R0^{hi}×CD45^{+/+}), which model the isoform expression of the A138G allele.

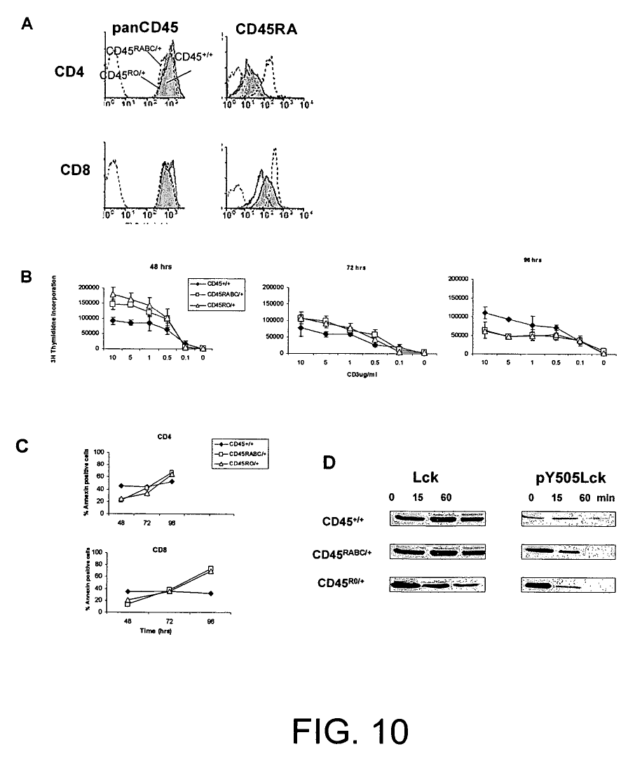

We analysed the expression of CD45 isoforms and total CD45 expression in CD4 and CD8 cells (FIG. 10A). The level of total CD45 expression is very similar in both CD4 and CD8 cells in CD45^{+/+}, CD45^{RABC/+} and CD45R0^{/+} mice. However staining with CD45RA indicates that the balance of different isoforms is altered in these CD45^{RABC/+} and CD45R0^{/+} mice due to the presence of the transgene.

Since the levels of CD45 expression are normal we next determined whether the alterations in the balance of isoforms would affect CD3/CD28 responses. Lymphocytes from lymph nodes of CD45^{+/+}, CD45^{RABC/+} or CD45R0^{/+} mice were activated with plate bound CD3/CD28. We observed differences between the response of the wild type CD45^{+/+} and the CD45^{RABC/+} and CD45R0^{/+} mice. In the first 48 hours CD45^{RABC/+} and CD45R0^{/+} cells show clearly increased proliferation, followed by a reduction at 96 hours compared to CD45^{+/+} mice (FIG. 10B). Because proliferative responses are a balance of cell division and cell death, we studied apoptosis during responses to CD3/CD28. FIG. 10C shows that the pattern of apoptosis is a mirror image of the proliferative responses, with the CD45^{RABC/+} and CD45R0^{/+} mice showing less apoptosis at 48 hrs, but an increase compared to CD45^{+/+} control cells at 96 hrs.

We assessed the levels of Lck and pY505Lck phosphorylation. Basal levels of Lck are the same in the three strains of mice, but pY505Lck is hyperphosphorylated in CD45^{RABC/+} and CD45R0^{/+}. However pY505LCK in CD45^{RABC/+} and CD45R0^{/+} cells shows greater dephosphorylation at 15 and 60 min following CD3/CD28 stimulation (FIG. 10D). The increased dephosphorylation correlates with the brisk initial proliferative response and suggests increased CD45 phosphatase activity in the CD₄₅^{RABC/+} and CD45R0^{/+} mice.

These results indicate clearly that the altered balance of isoforms in both CD45^{RABC/+} and CD45R0^{/+} mice does lead to altered magnitude and kinetics of proliferative responses compared to CD45^{+/+} mice.

Proliferative response of single isoform transgenic mice were also assessed using identical experimental techniques. The proliferative response of both CD45RABC^{hi} and CD45RO^{hi} lymph node T cells to CD3/CD28 was less than 30% of that of CD45^{+/+}. Neither of the low expressing lines CD45RABC^{lo} and CD45RO^{lo} were able to respond to CD3/CD28 (data not shown). Moreover, reduced CD45 phosphatase activity (assessed as the ability to remove inhibitory tyrosine phosphate from pY505Lck) was observed in cells expressing CD45RABC^{hi} and CD45RO^{hi} single isoforms, as compared to CD45^{+/+} cells (data not shown). Taken together these results indicate that a threshold level of single CD45 isoform expression is required for normal T cell receptor signalling and induction of proliferative responses and that cells expressing single CD45RABC or CD45RO isoforms at similar levels show identical patterns of response.

Cytokine Production in CD45Tg/+ Mice

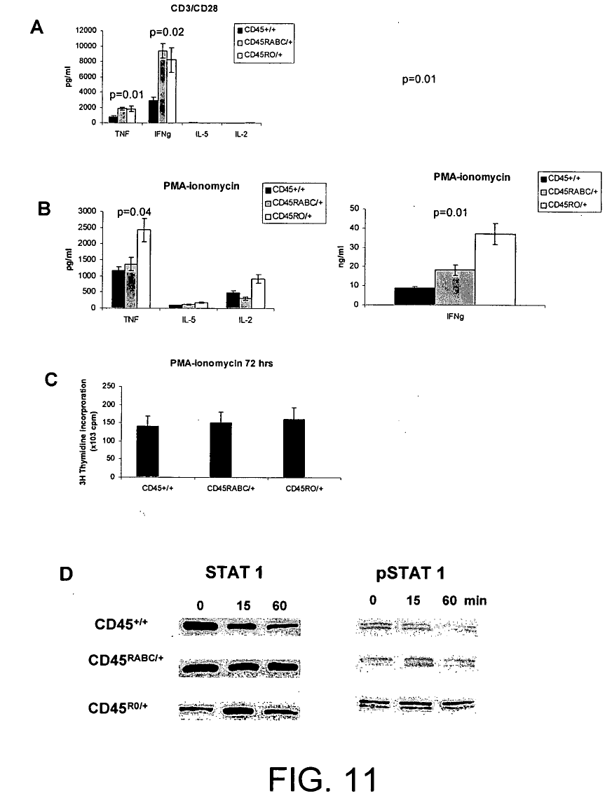

We next analysed cytokine production in CD45^{RABC/+} and CD45R0^{/+} mice after stimulation with CD3/CD28 or PMA-ionomycin. Both CD45^{RABC/+} and CD45R0^{/+} cells produced more TNFα and IFNγ compared to CD45^{+/+} in response to CD3/CD28 stimulation (FIG. 11A). Interestingly when stimulated with PMA-ionomycin the CD45R0^{/+} mice consistently showed the highest production of TNFα and IFNγ at 72 hrs (FIG. 11B). Increased cytokine production was also observed at 24 and 48 h after PMA-ionomycin stimulation (data not shown). Nevertheless proliferative responses to PMA-ionomycin are the same in all strains of mice (FIG. 11C).

It has been shown that PMA-ionomycin induced IFNγ production is dependent on signal transducer of activation of transcription 1 (STAT1) and that the Th1 specific transcription factor T-bet, that increases IFNγ production, is regulated by IFNγ signaling through STAT1 (Afkarian et al. Nat Immunol. 3: 549-557, 2002). We therefore measured the basal and phosphorylated levels of STAT1 following PMA-ionomycin stimulation. More phosphorylated STAT1 was detected in CD45^{RO/+} mice, providing an explanation for the increased IFNγ production in these mice.

EAE in CD45Tg Mice

Human CD45 variant alleles have been associated with autoimmune diseases. The C77G variant has been found with increased frequency in MS, while the A138G allele has been shown to be protective in Graves' disease. Since the CD45^{RABC/+} and CD45^{RO/+} mice have altered TCR signaling and cytokine production, we next tested whether this would lead to altered disease susceptibility or progression in a model of autoimmune disease. Experimental autoimmune encephalomyelitis (EAE) is a model for both MS in humans and the induction of autoimmune reactivity. We therefore used a Myelin Oligodendrocyte Glycoprotein (MOG) peptide to induce EAE in CD45^{+/+}, CD45^{RABC/+} and CD45^{RO/+} mice.

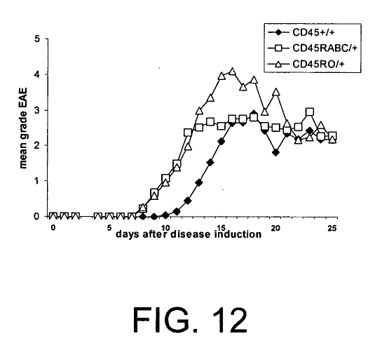

Both CD45^{RABC/+} and CD45^{RO/+} developed more severe disease compared to CD45^{+/+} mice with average total score of 2.2±0.8 and 2.5±1.1 respectively versus 1.6±1.1 in control CD45^{+/+} mice (FIG. 12). Disease onset was also earlier in CD45^{RABC/+} and CD45^{RO/+} mice (day 9.6±1.1 and 10.3±1.8 versus 12.6±2.4 in CD45^{+/+} mice). In all three experiments the CD45^{RABC/+} mice developed the disease first (day 9.6±1.1), while the signs of disease were more severe in CD45^{RO/+} mice (average maximal disease score 4.8±1.3 versus 3.5±0.9 and 3.1±1.1).

These results indicate that altering the balance of isoforms is important for immune function and that CD45^{RABC/+} and CD45^{RO/+} mice are more susceptible to induction of autoimmune disease in the EAE model.

Discussion

Here we report that the expression of combinations of CD45 isoforms affects immune function. Both CD45^{RABC/+} and CD45^{RO/+} mice show altered kinetics and magnitude of proliferative responses. The changes in signaling are complex, showing on the one hand more basal phosphorylated pY505Lck and on the other, following triggering through the TCR, more rapid and efficient dephosphorylation. The outcome of these changes is an altered TCR threshold reflected in the rapid proliferative responses of CD45^{RABC/+} or CD45^{RO/+} cells. As a result of the more vigorous TCR response, more TNFα and IFNγ are produced by CD45^{RABC/+} or CD45^{RO/+} cells.

The increased STAT1 phosphorylation in CD45^{RO/+} cells further indicates that CD45 isoform expression patterns affect not only cytokine production but also the response to cytokines. Here for the first time we show that an excess of the CD45R0 isoform preferentially induces TNFα and IFNγ production and a bias towards a Th1 response, indicating that the balance of isoforms affects both signaling for production of cytokines and the response to cytokines.

The most striking effect of altered CD45 isoforms expression is on the development of EAE. Both CD45^{RABC/+} and CD45^{RO/+} mice showed more rapid onset and increased severity of disease than CD45^{+/+} mice, with the most severe disease occurring in CD45^{RO/+} mice. TNFα and IFNγ have been shown to influence the severity of disease in this model in a complex manner. It is therefore not surprising that CD45^{RO/+} mice, which have altered regulation of TNFα and IFNγ production, show the most severe disease.

Here for the first time we have shown experimentally that altering the combination of CD45 isoforms dramatically affects immune function and disease severity in an autoimmune model. The data also show that the mechanism is an altered threshold for TCR signaling and altered cytokine production and response. This indicates that manipulating the patterns of CD45 expression or signaling pathways it modulates will be a useful therapeutic strategy. Mice with altered combinations of CD45 isoforms such as the CD45^{RABC/+} or CD45^{RO/+} transgenics described here, will provide models for future in depth studies of the underlying mechanisms.

### EQUIVALENTS

Those skilled in the art will recognize, or be able to ascertain using no more than routine experimentation, many equivalents to the specific embodiments of the invention described herein. Such equivalents are intended to be encompassed by the following claims.

All references disclosed herein are incorporated by reference in their entirety.

1. A method of screening a human subject for susceptibility to viral infection and/or pre-disposition to developing severe disease following viral infection, which method comprises screening for the presence or absence in the genome of the subject of one or more polymorphic variants or mutations in the gene encoding CD45 or of one or more polymorphic variants in linkage disequilibrium with or in close physical proximity to a polymorphic locus in the gene encoding CD45.

2. A method according to claim 1 wherein the mutation in the gene encoding CD45 is characterised in that subjects carrying at least one mutant allele exhibit altered CD45 splicing resulting in an increased proportion of cells expressing CD45RA and therefore a reduced proportion of single positive CD45RO+ T cells as compared to subjects not carrying a mutant allele, wherein subjects having at least one mutant allele are scored as being more susceptible to viral infection and/or more pre-disposed to developing severe disease following viral infection, as compared to subjects who do not carry a mutant allele.

3. A method according to claim 2 wherein the mutation in the gene encoding CD45 is the C77G mutation, wherein subjects having at least one 77G mutant allele are scored as being more susceptible to viral infection and/or more pre-disposed to developing severe disease following viral infection, as compared to subjects who do not carry a mutant allele, and/or the mutation in the gene encoding CD45 is the C59A mutation, wherein subjects having at least one 59A mutant allele are scored as being more susceptible to viral infection and/or more pre-disposed to developing severe disease following viral infection, as compared to subjects who do not carry a mutant allele, and/or the mutation in the gene encoding CD45 is the A54G mutation, wherein subjects having at least one 54G mutant allele are scored as being more susceptible to viral infection and/or more pre-disposed to developing severe disease following viral infection, as compared to subjects who do not carry a mutant allele.

4-5. (canceled)

6. A method according to claim 1 which comprises screening for the presence or absence in the human subject of a polymorphic variant or mutation in linkage or linkage disequilibrium with at least one mutation in the gene encoding CD45 selected from the group consisting of the C77G mutation, the C59A mutation and the A54G mutation, wherein subjects having at least one allele in linkage or linkage disequilibrium with the 77G mutant allele and/or the 59A mutant allele and/or the 54G mutant allele are scored as being more susceptible to viral infection and/or more pre-disposed to developing severe disease following viral infection, as compared to subjects who do not carry an allele in linkage or linkage disequilibrium with the 77G mutant allele and/or the 59A mutant allele and/or the 54G mutant allele.

7. A method according to claim 2 wherein the viral infection is infection with a virus selected from the group consisting of: human immunodeficiency viruses, HIV-1, Epstein-Barr virus, poliovirus, hepatitis B and hepatitis C virus.

8. A method according to claim 1 wherein the mutation in the gene encoding CD45 is characterised in that subjects carrying at least one mutant allele exhibit altered CD45 splicing resulting in an increase in the proportion of the T cell population carrying the CD45R0 isoform and lacking CD45RA expression as compared to subjects not carrying a mutant allele, wherein subjects having at least one mutant allele are scored as being less susceptible to viral infection and/or pre-disposed to developing less severe disease symptoms following viral infection, as compared to subjects who do not carry a mutant allele.

9. A method according to claim 8 wherein the mutation in the gene encoding CD45 is the A138G mutation, wherein subjects having at least one 138G mutant allele are scored as being less susceptible to viral infection and/or pre-disposed to developing less severe disease symptoms following viral infection, as compared to subjects who do not carry a 138G mutant allele.

10. A method according to claim 1 which comprises screening for the presence or absence in the human subject of a polymorphic variant or mutation in linkage or linkage disequilibrium with the Al 38G mutation in the gene encoding CD45, wherein subjects having at least one 138G mutant allele are scored as being less susceptible to viral infection and/or pre-disposed to developing less severe disease symptoms following viral infection, as compared to subjects who do not carry an allele in linkage or linkage disequilibrium with the 138G mutant allele.

11. A method according to claim 8 wherein the viral infection is infection with hepatitis B virus or hepatitis C virus.

12. A method of screening a human subject for an altered immune response capability, which method comprises screening for the presence or absence in said subject of a mutation in the gene encoding CD45, which mutation is characterised in that subjects carrying at least one mutant allele exhibit altered CD45 splicing resulting in an increase in the proportion of the T cell population carrying the CD45R0 splice variant but lacking CD45RA expression as compared to subjects not carrying a mutant allele, wherein subjects having at least one mutant allele are scored as having altered immune response capability.

13. A method according to claim 12 wherein the mutation in the gene encoding CD45 is the A138G mutation, wherein subjects having at least one 138G mutant allele are scored as having altered immunological response capability, as compared to subjects who do not carry a 138G mutant allele.

14. A method according to claim 12 wherein the altered immune response capability is a more protective response to infection by pathogenic substances or organisms, wherein subjects having at least one mutant allele are scored as exhibiting a more protective response to pathogenic substances or organisms than subjects not having a mutant allele: and/or the altered immune response capability is increased production of interferon-gamma by CD4 and/or CD8 T cells, wherein subjects having at least one mutant allele are scored as exhibiting increased production of interferon-gamma by CD4 and/or CD8 T cells as compared to subjects not having a mutant allele; and/or the altered immune response capability is an increase in the proportion of T cells having the activated, memory or effector phenotype, wherein subjects having at least one mutant allele are scored as exhibiting an increased proportion of T cells having the activated, memory or effector phenotype as compared to subjects not having a mutant allele.

15-16. (canceled)

17. The method of claim 12 for use in evaluating susceptibility of a human subject to autoimmune disease, wherein subjects having at least one mutant allele are scored as having altered immune response capability and therefore having reduced susceptibility to autoimmune disease, as compared to subjects not having a mutant allele; and/or for use in evaluating the likely severity of autoimmune disease symptoms in a human subject, wherein subjects having at least one mutant allele are scored as having altered immune response capability and therefore likely to exhibit less severe autoimmune disease symptoms, as compared to subjects not having a mutant allele; and/or for use in evaluating susceptibility of a human subject to viral infection, wherein subjects having at least one mutant allele are scored as having altered immune response capability and therefore having reduced susceptibility to viral infection as compared to subjects not having a mutant allele; and/or for use in evaluating the likely severity of disease symptoms following viral infection in a human subject, wherein subjects having at least one mutant allele are scored as having altered immune response capability and therefore likely to exhibit less severe disease symptoms following viral infection, as compared to subjects not having a mutant allele: and/or for use in evaluating susceptibility of a human subject to allergy or atopic disease, wherein subjects having at least one mutant allele are scored as having altered immune response capability and therefore less susceptible to allergy or atopic disease as compared to subjects not having a mutant allele.

18. (canceled)

19. The method according to claim 17 wherein the autoimmune disease is Graves' disease, Hashimoto's thyroiditis or Type I diabetes.

20-21. (canceled)

22. The method according to claim 17 wherein the viral infection is infection with hepatitis B virus or hepatitis C virus.

23. (canceled)

24. The method of claim 12 for use in predicting the likely response of a human subject to a vaccine optionally, wherein the vaccine is an anti-tumour vaccine.

25-26. (canceled)

27. A method of screening a human subject for susceptibility to viral infection and/or pre-disposition to developing severe disease following viral infection which comprises evaluating the pattern of CD45 mRNA expression in the subject or evaluating the pattern of CD45 protein expression in the subject, wherein the presence of an abnormal pattern of CD45 mRNA expression characterised by reduced splicing out of exon 4 of the CD45 mRNA and a quantitative decrease in amount of CD45RO transcript, or the presence of an abnormal pattern of CD45 protein expression characterised as a reduction in the proportion of T lymphocytes expressing the CD45R0 isoform but lacking CD45RA expression is taken as an indication that the subject is susceptible to viral infection and/or pre-disposed to developing severe disease following viral infection.

28. A method according to claim 27 wherein the abnormal pattern of CD45 mRNA expression is that associated with the presence of a 77G or a 59A mutant allele of the gene encoding CD54; or the abnormal pattern of CD45 protein expression is that associated with the presence of a 77G mutant allele of the gene encoding CD45, wherein detection of the abnormal pattern of CD45 mRNA expression is taken as an indication that the subject is more susceptible to viral infection and/or more pre-disposed to developing severe disease following viral infection, as compared to subjects who do not carry a 77G or 59A mutant allele, and wherein detection of the of abnormal pattern of CD45 protein expression is taken as an indication that the subject is more susceptible to viral infection and/or more pre-disposed to developing severe disease following viral infection, as compared to subjects who do not carry a 77G mutant allele.

29. (canceled)

30. A method of screening a human subject for susceptibility to viral infection and/or pre-disposition to developing severe disease following viral infection which comprises evaluating the pattern of CD45 mRNA expression or the pattern of CD45 protein expression in the subject, wherein the presence of an abnormal pattern of CD45 mRNA expression characterised by a quantitative increase in the level of expression of the CD45R0 transcript, or the presence of abnormal pattern of CD45 protein expression characterised by an increase in the proportion of T lymphocytes expressing the CD45R0 isoform but lacking CD45RA expression is taken as an indication that the subject is not susceptible to viral infection and/or is pre-disposed to developing less severe disease following viral infection.

31. A method according to claim 30 wherein the abnormal pattern of CD45 mRNA expression or the abnormal pattern of CD45 protein expression is that associated with the presence of a 138G mutant allele of the gene encoding CD45, wherein detection of the abnormal pattern of CD45 mRNA expression or abnormal pattern of CD45 protein expression is taken as an indication that the subject is less susceptible to viral infection and/or pre-disposed to developing less severe disease following viral infection, as compared to subjects who do not carry a 138G mutant allele.

32. A method of screening a human subject for an altered immune response capability, which method comprises evaluating the pattern of CD45 mRNA expression in said individual, wherein the presence of an abnormal pattern of CD45 mRNA expression characterised by a quantitative increase in the level of expression of the CD45R0 transcript is taken as an indication that the subject has an altered immune response capability. and/or pre-disposed to developing less severe disease following viral infection, as compared to subjects who do not carry a 138G mutant allele.

32. A method of screening a human subject for an altered immune response capability, which method comprises evaluating the pattern of CD45 mRNA expression in said individual, wherein the presence of an abnormal pattern of CD45 mRNA expression characterised by a quantitative increase in the level of expression of the CD45R0 transcript is taken as an indication that the subject has an altered immune response capability.

33. A method according to claim 32 wherein the abnormal pattern of CD45 mRNA expression is that associated with the presence of a 138G mutant allele of the gene encoding CD45, wherein detection of the abnormal pattern of CD45 mRNA expression is taken as an indication that the subject has an altered immune response capability, as compared to subjects who do not carry a 138G mutant allele.

34. A method according to claim 32 for use in evaluating susceptibility of a human subject to autoimmune disease and/or the likely severity of autoimmune disease symptoms in a human subject, wherein detection of the abnormal pattern of CD45 mRNA expression is taken as an indication that the subject has an altered immune response capability and therefore has reduced susceptibility to autoimmune disease and/or is likely to exhibit less severe autoimmune disease symptoms, as compared to subjects who do not exhibit the abnormal pattern of CD45 mRNA expression.

35-52. (canceled)
